(19) United States
(12) Patent Application Publication
(10) Pub. No.: US 2012/0034210 A1
(43) Pub. Date: Feb. 9, 2012

(54) COMPOSITIONS FOR POTENTIATING APOPOSIS SIGNALS IN TUMOUR CELLS

(75) Inventors: Pierre Vacher, Le Pian Medoc (FR); Patrick Legembre, Merignac (FR); Laurence Bepoldin, Saint Medard en Jalles (FR); Pierre Soubeyran, Bordeaux (FR); Anne-Marie Vacher, Le Pian Medoc (FR); Benjamin Chaigne-Delalande, Bordeaux (FR)

(73) Assignees: INSTITUT BERGONIE, Bordeaux (FR); UNIVERSITE VICTOR SEGALEN BORDEAUX 2, Bordeaux (FR)

(21) Appl. No.: 13/132,389

(22) PCT Filed: Dec. 4, 2009
(86) PCT No.: PCT/EP2009/066466

§ 371 Date: Oct. 11, 2011

Related U.S. Application Data
(60) Provisional application No. 61/119,767, filed on Dec. 4, 2008.

(30) Foreign Application Priority Data
Dec. 4, 2008 (FR) 08/58261

(51) Int. Cl. A61K 39/395; A61K 38/17; G01N 33/566; C12Q 1/32; A61P 35/02; G01N 33/577; C12Q 1/37; C12Q 1/02; A61P 35/00; A61K 38/23; C12Q 1/68

(52) U.S. Cl. 424/133.1; 514/11.9; 514/18.9; 424/173.1; 424/142.1; 435/7.23; 435/26; 435/6.1; 435/23; 435/29

(57) ABSTRACT

The present invention concerns a composition for potentiating formation of DISC (Death Inducing Signaling Complex) macro-complex and for inducing apoptotic signal mediated by death receptors in tumour cells comprising a therapeutically effective amount of an active agent selected among a hypocalcemia-inducing agent, a calcium channel inhibitor and a calcium chelator in association with a therapeutically effective amount of an anticancer agent inducing an apoptotic signal via death receptors Fas, TNF-R1, DR4 and/or DR5.

The present invention relates in its different aspects to new compositions and methods aiming to increase apoptosis in tumour cells, and in particular to increase the sensitivity of said cells to anticancer treatments. It is based on the discovery that the delivery of agents capable of modulating directly or indirectly intracellular calcium in combination with an anticancer agent inducing an apoptotic signal via death receptors Fas, TNF-R1, DR4 and/or DR5 allowed potentiating the pro-apoptotic effect of said agents significantly in tumor cells.

Programmed cell death termed apoptosis, is essential for homeostasis of organs and tissues. Human diseases including cancer and auto-immunity occur when apoptotic processes are impaired. Resistance to apoptosis is an essential issue in cancer research as it occurs both during tumorigenesis and tumor relapse following chemotherapeutic treatment. It is admitted that most of the anticancer agents induce apoptosis, raising the possibility that defects in apoptotic programs contribute to treatment failure. Thus, one of the main goals in oncology is to overcome resistance of tumor cells towards apoptosis. For decades, the hallmark of medical treatment for cancer has been cytotoxic chemotherapy. More than 70 different drugs are currently available and new ones are being developed all the time. These drugs target dividing cells, including cancer and non-cancer cells (e.g. lymphocytes, hair follicles, small intestinal epithelium). As a result, tumor-suffering patients experience side effects such as alopecia, gastrointestinal symptoms, lymphopenia and myelosuppression.

Although non-specific and large spectrum cytotoxic chemotherapies remain the treatment of choice for many malignancies, targeted therapies recently became a component of treatment for many types of cancer, including breast, colorectal, lung, and pancreatic cancers, as well as lymphoma, leukemia, and multiple myeloma. The two main types of targeted therapy are monoclonal antibodies, such as rituximab (anti-CD20 targeting B lymphocytes) or Herceptin (anti-HER2 over-expressed in 25 to 30% of mammary tumors), and specific pharmacologic inhibitors such as the BCR-Abl tyrosine kinase inhibitor Imatinib mesylate, which efficiently eliminates leukemic cells from Chronic Myelogenous Leukemia (CML).

A dramatic advance in the understanding of the molecular mechanisms of chemotherapy-mediated cell death came from a set of data demonstrating that the chemotherapeutic agent doxorubicin (anthracyclin) killed leukemic cell lines through the induction of the Fas-mediated signalling pathway. Following these observations, it was confirmed in various in vitro and in vivo models that numerous anti-tumoral agents eliminated cancer cells in inducing a cell death signal through the activation of the death receptor Fas.

Fas (also termed CD95/APO-1) is a transmembrane receptor belonging to the TNF (tumor necrosis factor) receptor family. Its intracellular region encompasses an 80 amino acid stretch called the Death Domain (DD) that is essential for the induction of the apoptotic signal. The receptor Fas is expressed ubiquitously whereas its cognate ligand, FasL, a membrane potein, possesses a restricted expression pattern. Indeed FasL is detected in the area of “immune privilege” such as the eyes and the testis where it impairs the access of effector cells of the immune system. This cytotoxic ligand is expressed de novo at the plasma membrane of activated T lymphocytes and NK (natural killer) cells where it plays a capital function in the elimination of tumor cells. FasL is also found at the surface of chemotherapy-treated tumor cells leading to the elimination of the malignant cells through an autocrine or paracrine process. Fas plays a pivotal role in homeostasis of the immune system and in the elimination of infected or transformed cells. Fas mutations or dysfunction of the Fas signaling pathway favors leukemogenesis and tumorogenesis of lymphomas and melanomas.

Two apoptotic signalling pathways have been identified: (i) the extrinsic pathway which triggers the death of the cells when membrane receptors are activated (TNFR1, Fas, DR3 or Tramp/Wsl1/Lard/Apo3. TrailR1 or DR4/Apo2. TrailR2 or DR5/Trick/Killer and DR6), and (ii) an intrinsic pathway which during intracellular stress, such as the accumulation of breaks in genomic DNA or an endoplasmic reticulum stress, mediates the release of apoptotic factors by the mitochondria (Kroemer G et al., Nat Med 2000. 6: 513-519). Connections exist between both these pathways and, in both situations, proteases called caspases are activated.

The Fas-mediated extrinsic pathway finally triggering apoptosis is illustrated in FIG. 1. The transduction effectors of the pro-apoptotic signal downstream of Fas have been partially identified and involve in particular the Fas/FADD/Caspase-8/c-FLIP macro-complex, also designated DISC or Death Inducing Signalling Complex. At a molecular level, upon binding of FasL, Fas DD aggregates, via homotypic interactions, the cytoplasmic adaptor protein Fas-Associated Death Domain protein (FADD), which in turn recruits proteases called caspase-8 and -10. The close vicinity of these initiator caspases favors their auto-cleavage and activation. A caspase-like protein termed c-FLIP (cellular FADD-like IL-1b-converting enzyme inhibitory protein) blocks the Fas-mediated cell death signal. c-FLIP_{L} shares extensive homology with the caspase-8 catalytic domain however its sequence lost the conserved amino acids essential for the catalytic activity. Therefore c-FLIP impairs the Fas-mediated cell death by competing with caspase-8 and -10 for the binding to FADD. Caspase-8 and 10 are considered as initiator caspases and the release in the cytoplasm of these cleaved caspases leads to the activation of the effector caspases (caspase-3, -6, -7), which in turn processes various substrates and trigger the dismantling of the cell structure.

While most anti-tumour treatments cause cellular death, probably by apoptosis and by activation of the death receptors, they however remain heavy treatments, difficult to tolerate by patients due to their non-specific cytotoxicity, and are still insufficient due to the occurrence of resistance mechanisms. Restoring and/or amplifying the apoptotic signalling in cancer cells thus present a major interest in cancerology.

The Applicants have herein evidenced that formation of the DISC macro-complex and induction of the pro-apoptotic signal via the “death receptors” mediated by anticancer agents, were significantly potentiated by reducing the free intracellular calcium concentration and/or by modulating the activity of calcium channels of the plasma membrane.

Hypercalcemia, regardless whether moderate to severe >3 mM, has been observed in 55% of hyperparathyroidia, in 30% of cancer and for 15% for other pathologies. It should be noted that in the case of neoplastic diseases, such as mammary carcinomae, lung and kidney cancers, prostate cancers, lymphomae or multiple myelomae, hypercalcemia is generally associated with the onset of bone metastases, with an acceleration of bone resorption as well as an increase in calcium retention by the kidneys. In addition to these phenomena, the neoplasic cells would secrete substances similar to PTH or parathyroid hormone related protein (PTH-rP) which not only stimulates the osteoclastic activity, but also would modifies absorption, excretion and resorption of calcium and phosphate ions. Consequently, 10-20% of the cancer patients have hypercalcemiae as the most severe metabolic diseases associated with neoplasic diseases. These patients are generally treated with hypocalcemia-inducing agents so as to reduce the complications resulting from bone metastases, and to increase their survival and quality of life.

Also, intracellular calcium is known to participate in cell signaling as a second messenger, whose message depends on its temporal features (i.e., duration, frequency), its spatial localization and its magnitude. The complexity of the intracellular calcium patterns accounts for its participation in a broad spectrum of cellular process such as proliferation, differentiation, migration and death. According to recent clinical observations, the role of calcium, which has long been considered as a catalyst of cell death, appears in fact more ambivalent. For instance, the use of calcium supplementation with a platinum-based chemotherapy drug alters the antitumoral effect in advanced colorectal cancer. Several anti-tumoral agents (e.g., doxorubicin, cisplatin, edelfosine, rituximab) have been described to cause elimination of malignant cells through the induction of the CD95 signal. However, no clinical studies have ever been conducted to assess whether the increase in intracellular ([Ca²⁺]ᵢ) and/or extracellular ([Ca²⁺]ₑ) concentrations of calcium prevented cell death by inhibiting the initial stages of the signal of death receptor such as CD95.

### SUMMARY OF THE INVENTION

The present invention relates to novel pharmaceutical compositions comprising the association of therapeutically effective amounts of an intracellular calcium modulator agent capable of reducing serum concentration of calcium, such as hypocalcemia-inducing agents, non permeant calcium chelators, or agents capable of reducing the intracellular calcium concentration such as channel-mediated calcium influx inhibitors, permeant calcium chelators with at least one anticancer agent capable of inducing a pro-apoptotic signal via death receptors. The novel therapeutic associations according to the invention are particularly useful for potentiating formation of the DISC complex, and for treating cancer and/or preventing cancer relapses.

The present invention also relates to the association of an intracellular calcium modulator agent capable of reducing the serum concentration of calcium such as hypocalcemia-inducing agents, non permeant calcium chelators, or an agent reducing intracellular calcium concentration such as channel-mediated calcium influx inhibitors or permeant calcium chelators with at least one anticancer agent capable of inducing a pro-apoptotic signaling via death receptors for potentiating formation of the DISC macro-complex and pro-apoptotic signal, as well as uses thereof for preparation of an anticancer treatment.

The present invention further relates to methods of treating cancer and/or cell proliferation, methods of preventing cancer relapses as well as to methods for sensitising tumour cells to the anticancer agents inducing an apoptotic signal via death receptors. According to a preferred embodiment, treated cancer patients are affected by primary tumour, hematopoietic cancer or solid tumor, and do not present any bone metastasis.

The present invention finally relates to a method of screening compounds capable of potentiating pro-apoptotic effect of the anticancer agents mediated by death receptors in tumour cells ex vivo.

### BRIEF DESCRIPTION OF FIGURES

FIG. 1: is a schematic representation of the induction cascade of the Fas-mediated apoptotic signal. FADD: Fas-associated via Death Domain; Cyt c: cytochrome C; Apaf-1: Apoptotic protease activating factor 1; DISC: Death-inducing Signalling Complex.

FIG. 2A: shows by Western blot technique and Fas immunoprecipitation, the formation of the DISC macro-complex in H9 cell line (lymphoma T-cell). The cells are non-treated (control), or pre-incubated with a calcium permeant chelator such as BAPTA-AM (10 μM), or a ionophore inducing the increase in intracellular calcium, i.e., ionomycine (1 μM) followed by activation for 15 minutes at 37° C. (15 min) or at 4° C. (0 min) in the presence of 1 μg/ml of anti-Fas APO1-3 agonist antibody. The cells are then lysed and Fas is immunoprecipitated and associated complex was analysed by Western blot.

FIG. 2B: represents the quantitative analysis by densitometry (ImageJ program) of the different components present in the DISC macro-complex during the immunoprecipitation of Fas in cells incubated with the reactants indicated previously.

FIG. 3A: illustrates the increase in percentage of cell death in different cellular models: the activated lymphocytes-T coming from healthy subjects (PBLs), T lymphocyte cell lines (Jurkat, H9, CEM, CEM-IRC) and B lymphocyte cell lines (SKW6.4, Raji, and BL2) were pre-incubated with the permeant calcium chelator BAPTA-AM at 1 μM then treated with the specified doses de FasL. Cellular death is quantified using an MTT test.

FIG. 3B: shows the expression of the Fas receptor on the surface of the B lymphomatous cell lines Raji and BL2 compared to isotypical labelling (negative control) using anti-Fas monoclonal antibodies and fluorescence intensity measurement by flow cytometry.

FIGS. 4A-D: show the apoptosis percentages in the Burkitt lymphoma cell lines, Raji and BL2, previously incubated in a medium containing or not (as indicated) the permeant chelator agent BAPTA-AM (1 μM), then treated with Rituximab or edelfosine. The % of apoptosis has been measured by the loss of the mitochondrial membrane potential Awm.

FIGS. 5A-C: show the effects of calcium ions chelation on the apoptotic signal induced by the DR4/DR5 apoptotic pathway. FIG. 5A shows the apoptosis % in the BL2 cell line (a) and in activated PBL (b), treated for 24 h with the TRAIL ligand with or without prior incubation (15 minutes) in a medium containing the permeant calcium chelator BAPTA-AM (1 μM) FIG. 5B shows the apoptosis % in the BL2 cell line (a) and in activated PBL (b), treated for 24 h with fluoxetine with or without prior incubation (15 minutes) in a medium containing the permeant calcium chelator BAPTA-AM (1 μM).

FIG. 5C shows the apoptosis % in the BL2 cell line treated for 24 h with MG132 (a) or with resveratrol (b) with or without prior incubation (15 minutes) in a medium containing the permeant calcium chelator BAPTA-AM (1 μM).

FIG. 6A: represents fluorometric measures (on a cell population using Indo-1 as calcium probe) of the intracellular calcium concentration in the Raji cell line. A 3 mM variation in the concentration of extracellular calcium induced a rapid increase (few seconds) in the intracellular calcium concentration (approx. 30 nM) (FIG. 6A—left graph). These cells present a smaller increase in the intracellular calcium concentration, in response to an extracellular application of 3 mM Ca²⁺, when they are pretreated with the calcium permeant chelator, BAPTA-AM (10 μM) (FIG. 6A—right graph).

FIG. 6B: shows the measures of the basal concentration of intracellular calcium in nM in relation to the concentration of extracellular calcium imposed (0 and 2 mM) in normal human B lymphocytes (left graph) and in tumoral B lymphocytes obtained from a lymphatic ganglion biopsy (right graph).

FIG. 6C: represents the measure of apoptosis % in the Raji cell line cultured in an RPMI medium added 10% foetal veal serum (FVS) containing 0.8 mM or 3 mM calcium, then treated with increasing concentrations of Rituximab. The apoptosis % has been measured by the drop of the mitochondrial membrane potential Δψm.

FIG. 6D: represents the % of apoptosis in the Raji cell line cultured in a defined medium containing 0.5 mM or 4 mM calcium, then treated with increasing concentrations of Rituximab. The apoptosis % has been measured by the loss of the mitochondrial membrane potential Δψm.

FIGS. 7A-B: illustrate the effect of 44 μM 2-APB, a calcium channel inhibitor, on the induction of apoptosis. 2-APB reduced the increase in the intracellular calcium concentration induced by Thapsigargin (left graph), a SERCA inhibitor that activates calcium influx, and by an increase in the extracellular calcium concentration from 0 to 5 mM (right graph). The 2-APB sensitizes drastically the Fas apoptotic signal in particular in leukemic T and B cells (FIG. 7B).

FIGS. 8 A-C: show the variation of intracellular calcium concentration measured with a Nikon microspectrofluorimeter (lndo-1 probe) following the addition of soluble CD95 ligand (or Fas ligand) in H9 (T lymphoma cell line) and Jurkat T leukemic cell line expressing the functional Fas receptor (FIG. 8A), in a Jurkat T leukemic cell line expressing a hemizygous mutated allele of CD95 (Jurkat-CD95-Q257K), in a mutated allele of the Fas receptor (Fas Q257K), as well as in various cell clones defective for caspase 8 (caspase-8−/−) or defective for the FADD adaptor (FADD−/−).

FIGS. 9 A and B: illustrate the effects of a pre-incubation of cells with either BAPTA-AM or the calcium channel inhibitor 2-APB on the basal intracellular calcium concentration (FIG. 9A), and on the formation of CD95 capping (FIG. 9B).

FIG. 10: shows by Western blot technique, Fas immunoprecipitation, and size exclusion chromatography, the formation of the CD95 aggregation upon a decrease in intracellular calcium concentration via a pre-treatment with BAPTA-AM in Jurkat cells.

FIGS. 11A-C: show by Western blot significant increase in FADD binding to CD95 upon addition of the IP3-R antagonist xestospongin C (FIG. 11A), induction of DISC formation upon addition of BAPTA-AM and 2-APB in H9 cell line, Jurkat leukemic T-cells, in activated peripheral blood T-lymphocytes (PBTs) (FIG. 11B), and the absence of FADD recruitment and DISC formation upon increase of intracellular calcium concentration by addition of ionomycin (FIG. 11C).

FIG. 12: shows caspase-8 activation by using proluminogenic substrate of caspase-8 upon down-modulation of [Ca²⁺]ᵢ upon addition of BAPTA-AM or 2-APB.

FIG. 13: represents the measure of apoptosis % in BAPTA-AM or 2-APB pre-incubated Raji H9, CEM, and SKW6.4 cell lines. The apoptosis % has been measured by the loss of the mitochondrial membrane potential Δψm.

FIGS. 14 A-D: illustrate the variation of the mitochondrial membrane potential and CD95-mediated apoptotic signal in various cells lines pretreated with BAPTA-AM (FIGS. 14 A-C), and in leukemic T-cell line Jurkat harboring a hemizygous mutated allele of CD95 (Jurkat-CD95Q257K) (FIG. 14D).

FIGS. 15 A-E: illustrate the variation of the intracellular Ca²⁺ concentration (nM) measured with a Nikon microspectrofluorimeter (Indo-1 probe) by increasing extracellular calcium concentration (FIG. 15A). FIG. 15B shows DISC formation in cells that are cultured in a medium supplemented with calcium (3 mM) as compared to cells incubated in a medium containing a lower amount of extracellular free calcium (1 mM). FIG. 15C shows by Western blot the enhanced recruitment of FADD as well as enhanced DISC formation in H9 cell lines treated with both BAPTA and EGTA (FIG. 15C).

FIGS. 15D and 15E show % of cell death of T and B-cell lines treated with either EGTA or BAPTA as compared to untreated cell lines (FIGS. 15D and 15E).

FIG. 16: illustrates the effects of Zoledronate in vitro and in vivo. FIG. 16A: Intracellular calcium concentration was measured in cell populations using indol-AM as fluorescent calcium probe and a spectrofluorometer Hitachi F2500. FIG. 16B: The percentages of apoptotic cells were determined in BL2, Raji, Jurkat cells by measuring the mitochondrial membrane potential (TMRM probe) in response to various concentrations of TRAIL, Rituximab (RTX) or FasL, as indicated, and in the presence or not of 10 μM Zoledronate (ZOL). FIG. 16C: Blood samples were taken from mice 3 days after physiological serum or Zoledronate injection (one injection per week, for 4 weeks, 4 mg/kg). FIG. 16D: Tumour mass (in mm³) following treatment with physiological serum (control), zoledronate alone (Zol, one injection per week, for 4 weeks, 4 mg/kg), Rituximab alone (RTX, one injection per week, for 4 weeks, 2 mg/kg), or a combination of the two drugs (ZRTX, one injection per week, for 4 weeks, RTX:2 mg/kg, Zoledronate: 4 mg/kg).

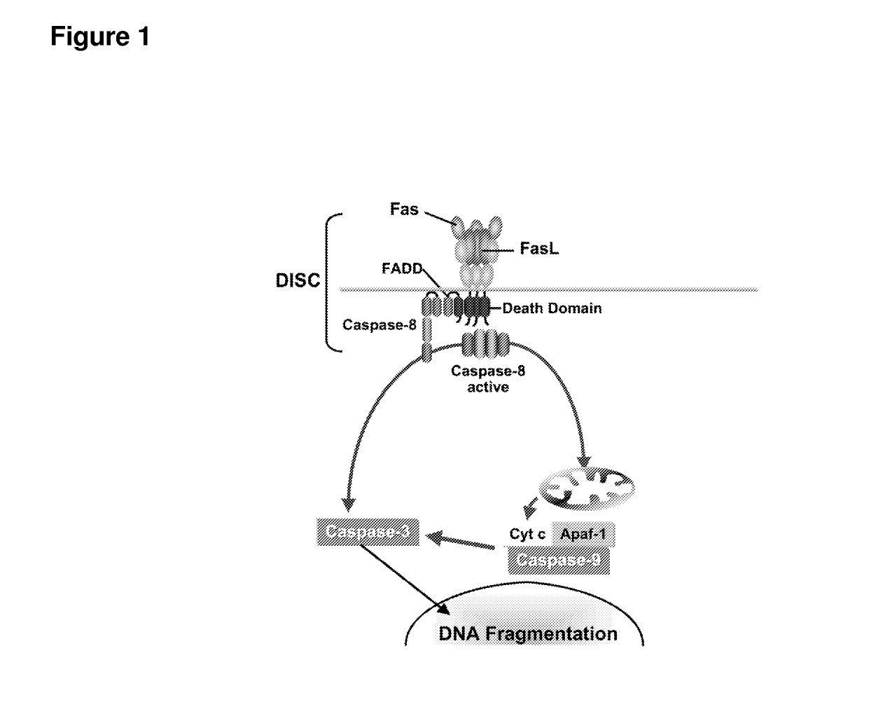

### DETAILED DESCRIPTION OF THE INVENTION

The present invention relates to novel compositions for potentiating the formation of the DISC macro-complex and inducing the apoptotic signal in tumour cells, comprising the association in therapeutically effective amounts of an intracellular calcium modulator agent capable of reducing the intracellular concentration of calcium and of at least an anticancer agent inducing an apoptotic signal via death receptors.

Within the framework of the present invention, the Applicants have surprisingly demonstrated that the tumoricide activity of the anticancer agents mediated by death receptors were significantly increased when the former were delivered in combination with at least one agent capable of reducing intracellular calcium and/or of modulating the activity of the calcium channels in tumour cells. The reduction of intracellular calcium has thus allowed potentiating formation of the DISC macro-complex as well as signaling of cellular death of particular chemotherapeutic agents.

Associations according to the invention increased considerably the efficiency of anticancer treatment. In other words, the therapeutic effect of the anticancer agents is unexpectedly potentiated by the delivery of an intracellular calcium concentration modulator agent.

Another major subsequent advantage offered by the associations according to the invention, concerns the possibility to use lower, but still efficient doses of anticancer agents, than those that are currently administered as standard chemotherapy, thereby reducing the risks of side effects, in particular cytotoxicity effect.

According to the invention, by intracellular calcium modulator agent is meant, any agent having a direct or indirect action on the calcium intracellular concentration in tumour cells. Preferably, these are according to the invention (i) agents capable of decreasing the intracellular calcium concentration, such as calcium channel inhibitors, and permeant calcium chelators, or (ii) agents whose activity aims at reducing the serum concentration of calcium such as hypocalcemia-inducing agents and chelators of extracellular calcium (non permeant). Indeed, the Applicants have shown that intracellular calcium concentration was strictly controlled by extracellular calcium concentration. Thus, the Applicants have also demonstrated that by decreasing extracellular calcium concentration, the apoptotic response, for instance to Rituximab, was potentiated in B lymphocyte cell lines.

The present invention thus relates to a composition for potentiating formation of DISC (Death Inducing Signalling Complex) macro-complex, inducing apoptotic signal mediated by death receptors in tumour cells comprising a therapeutically effective amount of an active agent selected among a hypocalcemia-inducing agent, a calcium channel inhibitor, and a permeant or non-permeant calcium chelator in association with a therapeutically effective amount of an anticancer agent inducing an apoptotic signal via death receptors Fas, TNF-R1, DR4 and/or DRS.

These intracellular calcium modulator agents are well-known in the field. By way of example, hypocalcemia-inducing agents may be chosen among biphosphonates, calcimimetic agents or calcitonine. Among the biphosphonates, palmidronate, zoledronate, etidronate, ibandronate or clodronate may be mentioned. Calcimimetic agents may be for example cinacalcet.

Calcium channel inhibitors are also well known in the field, and may be for example 2-Aminoethoxydiphenyl borate (2-APB), ML-9 or BTP-2.

The calcium chelators entail BAPTA (1,2-bis(o-amino phenoxy)ethane-N,N,N′,N′-tetraacetic acid), EGTA (glycol-bis(2-aminoethylether)-N,N,N′,N′-tetraacetic acid), EDTA (2-[2-(bis (carboxymethyl)amino)ethyl-(carboxymethyl)amino]acetic acid), or CDTA (trans-1,2-cyclohexane diamine-tetraacetic acid) which are non-permeant agents, or the corresponding permeant forms, that is to say BAPTA-AM (1,2-bis-(o-Amino phenoxy)ethane-N,N,N′,N′-tetraacetic tetra-(acetoxymethyl) acid Ester), derivatives of BAPTA-AM such as 5,5′-difluoro-BAPTA-AM (5,5′ F2 BAPTA), or 5-5′-dimethyl-BAPTA-AM, EGTA-AM, EDTA-AM and CDTA-AM. Preferred calcium chelator is BAPTA-AM ([1,2-bis-(o-Aminophenoxy)ethane-N,N,N′,N′-tetraacetic Acid Tetra-(acetoxymethyl) Ester]) which is known as a cell permeable compound not being able in its present form to chelate Ca²⁺. Once inside the cell, BAPTA-AM molecules are hydrolyzed by ubiquitous intracellular esterases, releasing a cell membrane impermeable Ca²⁺ chelator. Contrary to BAPTA-AM which specifically traps intracellular free calcium, unmodified non permeant BAPTA or EGTA chelates preferentially the extracellular free calcium (1-3 mM), whose amount is 10 000 fold more important than cytosolic calcium (−100 nM).

Alternatively, intracellular calcium modulator agent may be according to the present invention inhibitor of ORAI-1 channel, such as for example ML-9, BTP-2 or SKF9636.

By anticancer agents inducing a pro-apoptotic signal mediated by the death receptors is meant an active compound which, when delivered to a patient, induce the transmission of a pro-apoptotic signal via the death receptors, thereby eliminating tumour cells.

By death receptor is meant the TNFR1, Fas or Apo1/CD95, DR3 or Tramp/Wsl1/Lard/Apo3, TrailR1 or DR4/Apo2, TrailR2 and DR5/Trick/Killer and DR6 receptors which are involved in the extrinsic pathway of apoptosis signalling. Consequently, the associations of agents aiming to decrease the intracellular or extracellular concentration of calcium with anticancer agents inducing an apoptotic signal via death receptors Fas, TNF-R1, DR4 et/ou DR5 according to the present invention present a synergistic effect on the formation of the DISC complex and on the induction of the apoptotic signal in tumour cells.

To this regard, the Applicants have evidenced that stimulation of death receptor CD95 or Fas conducted to a rapid and transient increase in intracellular calcium, which prevented the induction of the apoptotic signal. Intriguingly, inhibition of this calcium peak and down-modulation of the intracellular concentration of calcium promoted the clustering of CD95 (CD95-CAP) and the recruitment of FADD to CD95. Finally, the Applicants showed that decrease in intracellular calcium enhanced the cytotoxic action of anti-tumoral drugs, thereby allowing elimination of malignant cells through the induction of the CD95-mediated apoptotic signal. Furthermore, the Applicants pointed out that down-modulation of calcemia may potentiate the death receptor signal triggered by either the immune system or chemotherapy.

Using leukemic cells lines and activated lymphocytes, the Applicants highlighted that CD95 engagement induced a rapid and transient increase in intracellular calcium occurring independently of the death domain-dependent structure called the DISC. While calcium has often been viewed as a potent catalyst of cell death, the Applicants evidenced surprisingly that this ionic flux displayed an anti-apoptotic function by preventing the formation of the DISC and more accurately, the recruitment of FADD. These findings pointed out that calcium targets the CD95 molecular ordering at the level of CD95 aggregation since [Ca²⁺]ᵢ decrease leads to the CD95L-independent clustering of CD95. Strikingly, death receptor aggregation remained insufficient for inducing cell death as long-term culture (up to 24 hours) of leukemic cell lines with BAPTA-AM or 2-APB drives CD95-CAP, but did not trigger the apoptotic signal.

It is known that although the release of calcium from endoplasmic reticulum store played a crucial role in the apoptotic signal triggered by intrinsic stimuli, modulation of the intracellular calcium does not exert a preponderant effect on the CD95-mediated apoptotic signal. In addition, the intensity, the frequency and the origin of the increase in intracellular calcium observed upon CD95 stimulation remain controversial. While certain observed a rapid PLCγ-dependent and IP3R-dependent increase in calcium, others measured a delayed augmentation of calcium upon CD95 stimulation. Controversy also exists about the origin of calcium, while certain authors showed that intracellular calcium increase only relies on the release of ER calcium stock, others underlined that both intracellular and extracellular calcium participate in the cytosolic calcium increase upon CD95 stimulation.

It has been further showed that calcium via its modulation of the Ca²⁺/calmodulin-dependent protein kinase II (CaMKII) was able to prevent the CD95-mediated apoptotic signal through the up-regulation of the anti-apoptotic factor c-FLIP. On the contrary, the Applicants did not observe any modification of the c-FLIP expression upon the down-modulation of calcium and furthermore, calcium targeted the FADD recruitment, which took place upstream the competitive inhibition of the caspase-8/-10 aggregation by c-FLIP. In addition, the use of calmidazolium, a calmodulin antagonist did not alter the CD95-mediated apoptotic signal and while the permeant CaMKII inhibitory peptide 281-309 was able to protect from the TRAIL-induced apoptotic signal, it did not affect the CD95 stimulation. It is noteworthy that the implication of CaMKII in the CD95-mediated apoptotic signal was investigated using the antagonist KN-93, which has also been reported to block the inositol 1,4,5-trisphosphate receptor (IP₃R) through a CaMKII-independent process. Altogether, these findings ruled out the implication of CaMKII in the recruitment of FADD by CD95.

Activation of PLCγ1 upon CD95 stimulation generates IP₃, which plays crucial role in inducing the intracellular calcium peak upon CD95 stimulation. While the link between PLCγ1 and CD95 remains to be uncovered but herein, the Applicants demonstrated that the death domain and major components of the DISC do not participate to this process. The calcium release from the endoplasmic reticulum store is followed by an entry of extracellular calcium through the SOC channels. The calcium increase observed in our experiments correspond to the sum of these processes, which both participate in the modulation of the apoptotic signal since both inhibition of IP₃—R and down-modulation of [Ca²⁺]ₑ improve the DISC formation and the subsequent induction of the apoptotic signal.

It is noteworthy that various chemotherapeutic agents have been reported to trigger the elimination of the malignant cells via aggregation of the death receptor and subsequent induction of the apoptotic signal. The Applicants further investigated whether the down-modulation of the extracellular concentration of calcium may promote the anti-tumoral effect of drugs killing malignant cells through the induction of the death receptor signal. These results could have great interest in oncology since we could envision that the decrease in calcemia in a physiologically acceptable spectrum may promote the cytotoxic action of either CD95L-expressing immune cells (activated T-lymphocytes and NK cells) or the CD95-dependent chemotherapeutic drugs.

The compositions of the present invention thus comprise in association with modulator of calcium concentration as described above, a therapeutically effective amount of an anticancer agent capable of inducing an apoptotic signal mediated by the death receptors Fas, TNF-R1, DR4 and/or DRS. Such anticancer agent is preferably selected among an anti-CD20 antibody, an anti-Fas antibody, an anti-TNF-R1 antibody, an anti-DR4 antibody, and an anti-DR5 antibody.

Alternatively, these anticancer agents may be selected among the FasL, the TRAIL, the soluble portions thereof and more generally all the ligands of the TNFR1, Fas (Apo1/CD95), DR3 or Tramp/Wsl1/Lard/Apo3, TrailR1 or DR4/Apo2, TrailR2 and DR5/Trick/Killer and DR6 death receptors.

Preferred agent is TRAIL (TNF-Related Apoptosis-Inducing Ligand), which has been described inter alia by Henson E S et al. (Leuk Lymphoma 2008. 49: 27-350). The TRAIL belongs to the TNF family and binds to DR4 or DR5 death receptors, thereby inducing a cell death signal comparable to that of the Fas receptor. The Applicants have showed in the Examples below that TRAIL presented in association a powerful tumoricide action on leukemic cells or on cells derived from lymphomae. Also, as showed in the Examples, induction of cell death by adding the TRAIL was also potentiated by calcium chelation.

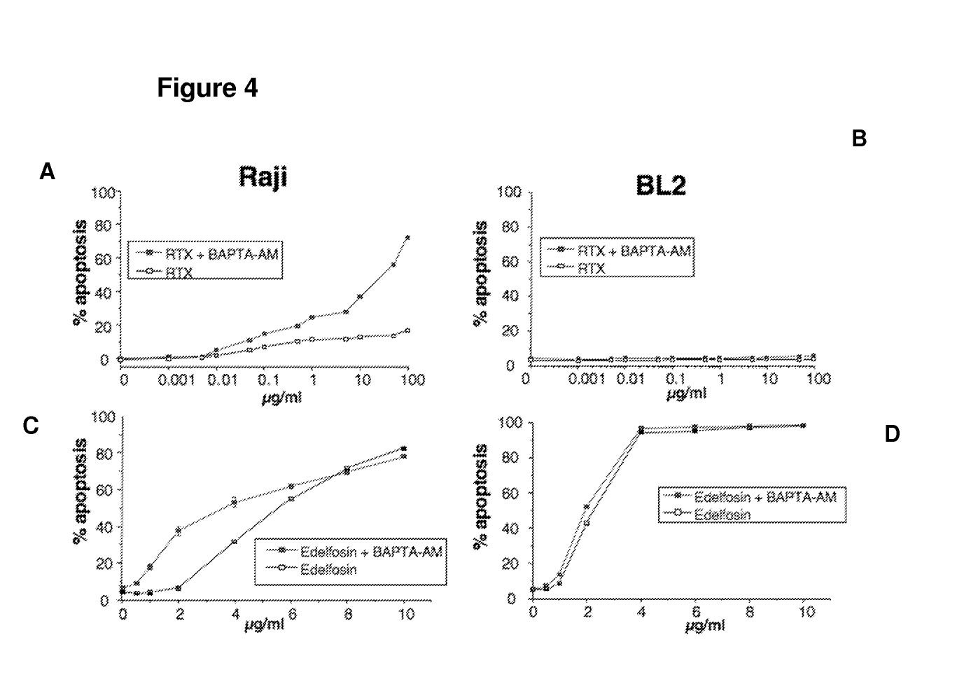

Among monoclonal antibodies, anti-CD20 antibodies such as Rituximab or Rituxan®, GA-101, ofatumab, LFB-R603, or Veltuzumab may be mentioned. Rituximab and its dependent Fas-tumoricide activity have been reported inter alia by Stel A J et al. (J Immunol 2007. 178: 2287-2295) and Vega, M. I. (Oncogene 2005. 24: 8114-8127). The Applicants have in particular demonstrated that the addition of Rituximab to B lymphocytes acted via Fas apoptotic signalling (FIGS. 4A-B).

Other anticancer agents capable of inducing a pro-apoptotic signal via death receptors which may be used in synergistic associations according to the present invention include proteasome inhibitors such as for example MG132; histone-deacetylase inhibitors (HDACi) such as for example trichostatin A, depsipeptides, suberoylanilide hydroxamic acid (SAHA), LAQ824, valproic acid, and benzamide; reverse transcriptase inhibitors such as for example Efavirenz; hypoglycaemic agents such as metformin and benfluorex; selective serotonin reuptake inhibitors such as fluoxetine, sertraline, paroxetine, and citalopram; tricyclics such as imipramine; thalidomide such as lenalidomide, actimid or pomalidomide; ether lipids such as edelfosine ilmofosine, and perifosine; and polyphenols such as resveratrol.

For more detail, the skilled person in the art may refer to the manual published by the French Association of Therapeutic Chemistry Teachers entitled “treatise on therapeutic chemistry, Vol. 6, Antitumoral medications and perspectives in treatment of cancers, TEC & DOC publishers, 2003”.

Finally, according to another aspect, the present invention relates to synergistic associations in therapeutically effective amounts of at least one intracellular calcium concentration modulator agent and of at least one phosphatidylinositol-3 kinase (PI3K) signalling pathway inhibitor. According to the present invention, these antitumoral associations enable to sensitise tumour cells to chemiotherapy significantly. Preferably, the PI3K signalling pathway inhibitor agent is edelfosine, LY294002 or wortmannin. More preferably still, the inhibitor used in the associations according to the present invention is edelfosine and its derivatives which is among others described by Beneteau M. et al. (Mol Cancer Res 2008. 6: 604-613). These inhibitors seemed to act on the Fas redistribution in lipid rafts (Wymann M P et al. Trends Pharmacol Sci 2003. 24: 366-376).

The Applicants have besides demonstrated that type II calmodulin kinase (CaMKII) was probably not involved in the modulation of the formation of DISC and/or in potentiating the pro-apoptotic signal according to the present invention.

According to the invention, the associations or composition as described above which are useful for potentiating formation of DISC (Death Inducing Signalling Complex) macro-complex and for inducing apoptotic signal mediated by death receptors in tumour cells may be administered to cancer patients in a therapeutic dose sufficient so as to effect a tumour growth reduction, such as for example in the case of B lymphoma tumours, prostate cancer tumours or breast cancer tumours, ranging from 10 to 90%; 15 to 90%, 20 to 90%; 25 to 90%; 30 to 90%; 35 to 90%; 40 to 90%; 45 to 90%; 50 to 90%; 55 to 90%; 60 to 90%; 65 to 90%; 70 to 90%; 75 to 90%; 80 to 90%; or 85 to 90%.

Preferably, patients which are treated are affected by primary tumours without any occurrences of metastasis.

According to the invention, an active agent selected among a hypocalcemia-inducing agent, a calcium channel inhibitor, or calcium chelator may be administered with an anticancer agent inducing an apoptotic signal via death receptors Fas, TNF-R1, DR4 and/or DR5 are simultaneously, separately or sequentially.

Simultaneous administration is intended to mean delivering both compounds of the composition according to the invention in a single pharmaceutical form. Separate administration means the delivery, at the same time, of both components of the composition in distinct pharmaceutical forms. Sequential administration means successive delivery of both compounds of the composition according to the invention, each in a distinct pharmaceutical form.

According to a preferred delivery protocol, the active agent selected among a hypocalcemia-inducing agent, a calcium channel inhibitor, or calcium chelator may be administered to a patient prior to the anticancer agent. It may be delivered a few days before, for instance between 1 and 10 days prior to the delivery of the anticancer agent. Also, the doses of calcium concentration modulator agent delivered to cancer patient are lower than those which are conventionally administered in the case of treatments of metabolic diseases associated with neoplasic diseases.

In a second embodiment, the present invention also relates to a pharmaceutical composition comprising as active principle the association or the composition as described above, preferably added with an excipient and/or a pharmaceutically acceptable vehicle. In the present description, by pharmaceutically acceptable vehicle is meant a compound or a combination of compounds used in a pharmaceutical composition which does cause any side reactions and which enables for instance easier delivery of the associations, increases its lifetime and/or its efficiency in the system, increases its solubility in solution or still improves its shelf life.

These pharmaceutically acceptable vehicles are well-known and may be adapted by a skilled person in the art according to the nature and the mode of delivery of the selected agent(s).

Preferably, these compounds delivered systemically, in particular intravenously, intramuscularly, intradermically, intraperitoneally, sub-cutaneously or orally.

The optimal mode of delivery, posologies and galenic forms may be determined according to the criteria generally taken into account when establishing a treatment and adapted to each patient as for example the age of the body weight of the patient, the severity of his general condition, tolerance to treatment and side effects.

The present invention also relates to the use of the associations or compositions as previously above for the preparation of a drug for treating cancer and/or for preventing cancer relapses. Such cancers are for instance colon, breast, prostate, lung (small cell and non-small cell), ovary, pancreas, kidney, brain, blood cell (lymphomae and leukemiae) and liver cancers. Preferably, cancers which are treated by the present compositions are primary cancers which do not show any occurrences of metastasis.

The present invention further relates to a method for treating, preventing and/or sensitising tumour cells comprising administering therapeutically effective dose of the composition as described above. Among the cancers which may be treated, colon, breast, prostate, lung (small cell and non-small cell), ovary, pancreas, kidney, brain, blood cell (lymphomae and leukemiae) and liver cancers may be mentioned preferably. Method of treating comprises administering to a patient the composition as described above in a therapeutic dose sufficient to reduce the volume and the growth of the tumour in particular in the case of B lymphoma tumours, prostate cancer tumours or breast cancer tumours, with rates ranging from 10 to 90%; 15 to 90%, 20 to 90%; 25 to 90%; 30 to 90%; 35 to 90%; 40 to 90%; 45 to 90%; 50 to 90%; 55 to 90%; 60 to 90%; 65 to 90%; 70 to 90%; 75 to 90%; 80 to 90%; or 85 to 90%.

The composition of the present invention may further comprise additional anti-tumoral agents capable of preventing or of inhibiting DNA, RNA and/or protein synthesis, such as for instance daunorubicine, idarubicine, valrubicine, mitoxantrone, dactinomycine, mithramycine, plicamycine, bleomycine, and procarbazine; or with immunomodulators which stimulate the immune system, i.e., NK cells and activated T lymphocytes which express FasL and TRAIL, such as interferons; interleukins such as aldesleukin, OCT-43, denileukin diflitox or rinterleukin-2; tumour necrosis factors such as tasonermin; or other types of immunomodulators such as lentinan, sizofuran, roquinimex, pidotimod, pegademase and thymopentine.

In addition, compositions according to the present invention may comprise antibodies having an anti-tumour activity. By way of non limiting examples, the anti Her2/neu (Herceptin), anti-EGFR (Erbitux) or still anti-IGF-IR antibodies may be mentioned.

According to a last embodiment, the present invention relates to a method of screening compounds capable of potentiating the pro-apoptotic effect of anticancer agents involving death receptors in tumour cells. The method of screening comprises contacting tested compounds to cells which are obtained from biopsies or from cancer cell lines, and assessing formation of the DISC macro-complex in presence and in absence of a modulator agent of the intracellular or extracellular calcium concentration, thus indicating selective potentiation. Preferred modulator agent of the intracellular or extracellular calcium concentration may be selected among a hypocalcemia-inducing agent, a calcium channel inhibitor and a calcium chelator.

Percentages of cell death are then assessed in the different treatment conditions of cell lines. More accurately, the induction of apoptosis may be monitored for instance by measuring the mitochondrial transmembrane electrical potential, the membrane permeability, the DNA fragmentation, the cell morphology, by Western blot or by measuring caspase activity.

Other characteristics and advantages of the invention appear in the following Examples and the Figures.

### EXAMPLES

### Example 1

### Cell Lines

The leukemic T-cell lines Jurkat, CEM and H9, the EBV (Epstein Barr Virus)-transformed lymphoblastoid B-cell line SKW 6. 4, the Burkitt lymphomae RAJ1 and BL2 cell lines originated from the ATCC (American Type Culture Collection). They were grown in an RPMI 1640 medium (Roswell Park Memorial Institute) complemented with 8% foetal veal serum (FVS, Sigma) (decomplemented for 30 minutes at 56° C.) and 2 mM L-glutamine (Gibco). The cells were grown in a humid incubator at 37° C. and 5% CO₂. The Fas-deficient CEM cell line (CEM-IRC) was allowed to grow for several generations in a FasL-containing medium and the resistant cells were cloned, then on the basis of the Fas expression, a deficient clone was isolated. The Jurkat clone Q257K was obtained from the Jurkat lineage which was grown for 3 weeks in the presence of a dose of 200 ng/ml agonist anti-Fas antibody (clone 7C11) corresponding to twice the dose enabling to reach the plateau of the maximum of cell death of the parent cells. Jurkat T leukemic cell lines which are deficient for caspase 8 or FADD originated from the ATCC.

### Example 2

### Activated T lymphocytes

The PBMCs (Peripheral Blood Mononuclear Cells) of healthy subjects were obtained after centrifugation in a Ficoll gradient, then monocytes/macrophages were eliminated by a one-hour adherence step on a culture flask. The PBLs (peripheral blood lymphocytes) thus isolated were activated for 20 hours with 1 μg/ml PHA-L (Phytohemagglutinin type L) then washed and stimulated for 6 days with 50 U/ml IL2 (Interleukine-2) in a culture medium consisting of RPMI, containing 10% human serum, 2 mM L-glutamine, 1.10⁵ units of penicillin, 1.10⁵ μg streptomycin (Gibco). A flow cytometry analysis of the CD3 and CD19 membrane markers was conducted after Ficoll for analysing the initial distribution of the B and T-cell populations.

### Example 3

### Reactants and Antibodies Used

The FasL (gp190-CD95L) was generated by Legembre P. et al. (J Immunol 2003. 171: 5659-5662). The soluble recombinant human TRAIL (TNF-related Apoptosis-Inducing Ligand) comes from Alexis Biochemicals Covalab (Villeurbanne, France).

The anti-Fas 7C11 (IgM) and APO1-3 (IgG3) agonist antibodies were obtained respectively from BD-Biosciences (Franklin Lakes, USA) and Alexis Biochemicals Covalab.

The antibodies used for the Western blot were the C20 anti-Fas human (Santa Cruz) and the HRP-coupled anti-rabbit goat polyclonal secondary antibody (Zymed, San Francisco, USA). The anti-Fas used for flow cytometry markers is the DX2 clone (IgG1). The DX2 were obtained from BD Biosciences, the mouse anti-IgG goat secondary antibodies were coupled either with phycoerythrin (PE) for flow cytometry or with Alexa555 fluorochrome (Invitrogen, Carlsbad, USA) for confocal microscopy.

Edelfosine and etoposide were obtained from Calbiochem (VWR International, Fontenay-sous-Bois, France),

Rituximab was obtained from Bergonié Institute.

BAPTA-AM, 2-APB and calmidazolium were obtained from Calbiochem (Merck Chemicals Ltd., Nottingham, UK). The permeant CaMKII inhibitory peptide (281-309), xestospongin C, BAPTA, EGTA, DAPI and DiOC6 were purchased from Sigma-Aldrich.

The anti-LIF isotype-matched negative control 1F10 (IgG) mAb and the anti-CD95L 10F2 were generated in the laboratory. Anti-caspase-8 (C15) and anti-CD95 mAb (APO1-3) were purchased from Axxora (Coger S. A., Paris, France). Anti-human CD95 Anti-human FADD mAb (Clone 1) were obtained from BD Biosciences (Le Pont de Claix, France). The anti-CD95 mAb (C20) was obtained from Santa Cruz Biotechnology (Santa Cruz, Calif., USA). The anti-human ORAI-1 and STIM-1 (extracellular epitope) rabbit polyclonal antibodies were obtained from Alomone Labs (Jerusalem, Israel).

### Cell Markings and Flow Cytometry Analysis

This technique allowed detecting the presence of a protein on the cell surface. All the steps took place at 4° C. so as to reduce membrane proteins endocytosis and loss of cell labelling.

Plasma membrane of the cells (10⁶ cells per marking) was saturated for 5 minutes in 1 ml PBS containing 1% (w/v) BSA (bovine serum albumin) and 1% (v/v) foetal veal serum (FVS). This solution was used for washings and antibody dilutions.

The cells were incubated for 30 minutes with 50 μl primary antibody (anti-Fas DX2 clone at 10 μg/ml), washed twice with the PBS/BSA/FCS solution, then incubated with 50 μl of a secondary antibody coupled with phycoerythrin for 30 minutes. After 3 washings, the cells were re-suspended in 200 μl PBS/BSA/SVF and analysed immediately by flow cytometry (FACsCalibur, BD Biosciences). The fluorescence intensity was analysed with the Cellquest software.

### Example 4

### Measurements of Intracellular Ca²⁺ Concentrations on Single Cell or Cellular Population

### Example 4.1

### Measurements on Single Cell

Principle: [Ca²⁺]ᵢ was measured by microspectrofluorimetry using a calcium sensitive fluorescent probe: indo-1. When excited at 360 nm wavelength, the so-called monoexcitation/double emission indo-1 probe transmitted back in a spectrum ranging from 400 to 500 nm. Binding of Ca²⁺ to indo-1 caused displacement of the emission spectrum after excitation. The emission maximum in the unbound form was located around 480 nm. The emission of the complexed form was around 405 nm. An increase of calcium concentration thus caused the emission to increase to 405 nm and the emission to decrease to 480 nm. The ratio of both fluorescence intensities allowed to be independent from the quantity of the probe and to determine the [Ca²⁺]i according to the Grynkiewicz equation:

[Ca²⁺]ᵢ(nM)=Kd.β×(R−Rmin)/(Rmax−R)

with: Kd: dissociation constant of indo-1
β: fluorescence ratio at 480 nm in absence of and in saturation of calcium
R: 405/480 ratio measured after correction of autofluorescence
Rmax: F405/480 ratio measured when indo-1 is saturated with calcium
Rmin: F405/480 ratio measured when indo-1 is free of calcium

Rmax, Rmin and Kd. β were determined by combining electrophysiology (patch clamp) and spectrofluorimetry. Rmax was determined by placing in the patch pipette a medium containing 10 mM CaCl₂. Rmin was obtained by placing as an intrapipette solution a calcium-deprived medium (10 mM EGTA). With a known concentration of free calcium in the solution of the patch pipette (300 nM, calcium mixture+EGTA) and knowing Rmin and Rmax, it was possible to calculate the value of the Kd. β product.

Protocol: The cells were incubated in the dark for 30 minutes at room temperature in a HBSS solution (Hank's Balanced Salt Solution: NaCl 142.6 mM; KCl 5.6 mM; Na₂PO₄ 0.17 mM; KH₂PO₄ 0.22 mM; glucose 5.6 mM; NaHCO₃ 4.2 mM) added with 1 μM acetoxymethylester indo-1 (indo-1/AM), pluronic acid (0.02%) and Ca²⁺ (at required concentration). The cells were then rinsed with HBSS (800 rpm centrifugation for 2 minutes) and placed in an observation chamber on a glass coverslip. Cells were observed in phase contrast using an epifluorescence inverse microscope (Nikon). The excitation light is supplied by a Xenon lamp (100W). A system of dichroic mirror and of interdifferential filters allowed continuous measuring of the fluorescence emitted at 405 and 480 nm. An analogue divider displayed the F405/F480 ratio continuously and translated said ratio into calcium variation from the Grynkiewicz equation (given above). The substances to be tested were applied using a glass pipette positioned at some twenty μm of the studied cell and connected to a pneumatic system.

### Example 4.2

### Measurements on Cellular Population

The principle as well as the loading of the cells were identical with the measurement on single cells. In such a case, approx. 40,000 cells were placed in a quartz tub under constant agitation. The tub was then placed in a Hitachi spectrofluorimeter whereof the excitation monochromator was set to 350 nm. The fluorescence emitted by the calcium probe Indo1 was captured and measured alternately (every second) at 405 and 480 nm by a photomultiplier. The signals were transmitted to a computer via an analogue/digital converter. So as to be able to apply the Grynkiewicz equation (see above) and translate the fluorescence ratios measured in calcium values, the calibration parameters were entered into the software. The substances to be tested were applied directly in the tube under constant agitation.

### Example 5

### Measurement of Cell Death

2 to 4.10⁴ cells were deposited in wells in a 96-well plate. The cells were incubated, in the presence of the different reactants for the times specified, at 37° C. in a final volume of 100 or 200 μl.

### Example 5.1

### MTT Measurement of Cell Viability

The MTT test enabled to measure the activity of a mitochondrial enzyme, the dehydrogenase succinate which transforms MTT (or 3-(4,5-dimethylthiazol-2-yl)-2,5-diphenyltetrazolium) bromide originally yellow in colour and soluble, into blue formazan crystals, insoluble in aqueous phase. Upon completion of the different treatments, the living cells were quantified by adding in each well 15 μl PBS (Phosphate Buffered Saline) containing 5 mg/ml MTT. After 4 h incubation at 37° C., the formazan crystals were dissolved in 105 μl of a solution containing 95% isopropanol and 5% formic acid. Spectrophotometric reading of absorbance was done at 570 nm. The measurement of the optical density is directly proportional to the number of living cells and the percentage of cell death was calculated with the following formula: 100-[(DO treated cells/DO non-treated cells)*100].

### Example 5.2

### Measurement of DNA Fragmentation

The cells were permeabilised by using a solution containing a low amount of detergent and their DNA-content was estimated by propidium iodide labelling. Propidium iodide was a fluorescent molecule which was interposed in double-strand DNA. The permeabilised cells having undergone the apoptotic process released the cleaved DNA which diffused through the nuclear and cellular pores. As a consequence, the apoptotic population displayed a lower DNA quantity than living cells and is called sub-G1 or aneuploid population.

Treated cells (2.10⁵ per well) were incubated for 4 hours at 4° C. with 200 μl of a buffer containing 0.1% (w/v) sodium citrate and 0.1% (v/v) Triton-X-100 with 50 μg/ml propidium iodide (Sigma). The fluorescence emitted by propidium iodide present in the cell was measured by flow cytometry since it is excitable by Argon laser (488 nm) and its emission wavelength is 637 nm.

### Example 5.3

### Measure of the Loss of Mitochondrial Membrane Electrical Potential

The apoptotic cells were identified in the cellular populations by measuring mitochondrial de-polarisation. The decrease in potential of the mitochondrial internal membrane (Δψm), a feature of apoptosis, was measured using the tetramethylrhodamine methyl ester fluorescent probes (λex=488 nm, λem=525 nm) (TMRM, Sigma) or 3,3′-dihexyloxacarbocyanine iodide (DiOC6).

These potentiometric probes allowed monitoring the variations of the mitochondrial membrane potential. In basal conditions, the mitochondrial membrane was hyperpolarised (−180 mV) and the probes accumulate into the mitochondriae (the cells were fluorescent), whereas in apoptotic conditions, the mitochondrial membrane was depolarised and the probes do not accumulate in the mitochondriae (the cells lost its fluorescence).

Following cell treatment with the different drugs, the former were placed in the presence of TMRM or DIOC6 at 10 nM concentration, for 20 minutes at 37° C. then the fluorescence of the cells was analysed by flow cytometry.

### Example 6

### DISC, Cell Lysate, Western Blot

Cells were incubated with 1 μg/ml APO1-3 for 30 minutes at 4° C. After washing, cells were incubated for 15 minutes at 4° C. (0 minutes) or at 37° C. (15 minutes) and then lysed for 30 minutes at 4° C. in a lysis buffer (25 mM HEPES pH 7.4, 1% Triton X-100, 150 mM NaCl, 2 mM EDTA, a cocktail including protease inhibitors (Sigma)). The lysed cells were then centrifugated for 15 minutes at 15000 rpm in order to eliminate genomic DNA, and the supernatant was kept. The sepharose beads which were coupled with protein A were added to the lysate and the mixture was incubated for 2 hours. The beads were recovered, washed intensively and the immunoprecipitated proteins were re-suspended in a denaturing and reducing buffer (0.01M Tris-HCl pH6.8, 10% glycerol (v/v), 85 mM sodium dodecyl sulfate (SDS), 5% (v/v) 3-mercaptoethanol and 0.005% (w/v) bromophenol blue) then heated at 100° C. for 5 minutes. The samples were deposited in an acrylamide/Bisacrylamide gel and separated by electrophoresis. The proteins were transferred from the gel into a nitrocellulose membrane using a semi-dry medium transfer technique for 2 hours at constant amperage (0.8 mA/cm²). The transfer buffer was composed of 25 mM Tris, 192 mM glycin, 0.1% SDS, 20% ethanol. After transfer, the nitrocellulose membrane was saturated with proteins for 30 minutes with a TBST buffer (50 mM Tris pH=8, 0.15M sodium chloride, 0.05% (v/v) Tween-20) containing 5% (w/v) skimmed powdered milk (TBSTM). After washing with TBST, the membrane was incubated for 2 hours with the primary antibody in TBST. The membrane was washed intensively then incubated for one hour with TBST containing the secondary peroxidase-coupled antibody. The presence of the protein of interest was revealed with an ECL solution (Enzyme Chemoluminescence, Pierce). This solution contained a substrate metabolised by peroxidase to yield a luminescent compound, which allowed marking a radiographic film (Amersham).

### Example 7

### Fluorescence Confocal Microscopy

The cells were left to adhere on a slide pre-treated with poly-L-lysin (ESCO, VWR) for 5 minutes then incubated with the different reactants. The cells activated with the anti-Fas APO1-3 agonist antibody were washed and marked directly with the anti-mouse secondary antibody coupled with Alexa555 fluorochrome. The cells incubated with Rituximab (anti-CD20) were washed in a cold PBS buffer then fixed with a PBS/4% PFA (paraformaldehyde) solution for 15 min. After washing, the cells were incubated with the anti-Fas antibody (clone DX2) (30 min at 4° C.) and then with the secondary antibody (30 min at 4° C.) as describes previously. The cells were washed in PBS medium then the slide was dried and the cells were placed in a mounting medium, Fluoroprep (Biomerieux) and analysed by fluorescence confocal microscopy (LSM SP5, Leica, Germany) with an x63 zoom. The DAPI (200 ng/ml) allowed colouring the nuclei.

### Example 8

### Evidence of a Potentiating Effect of the Formation of the DISC Macro-Complex

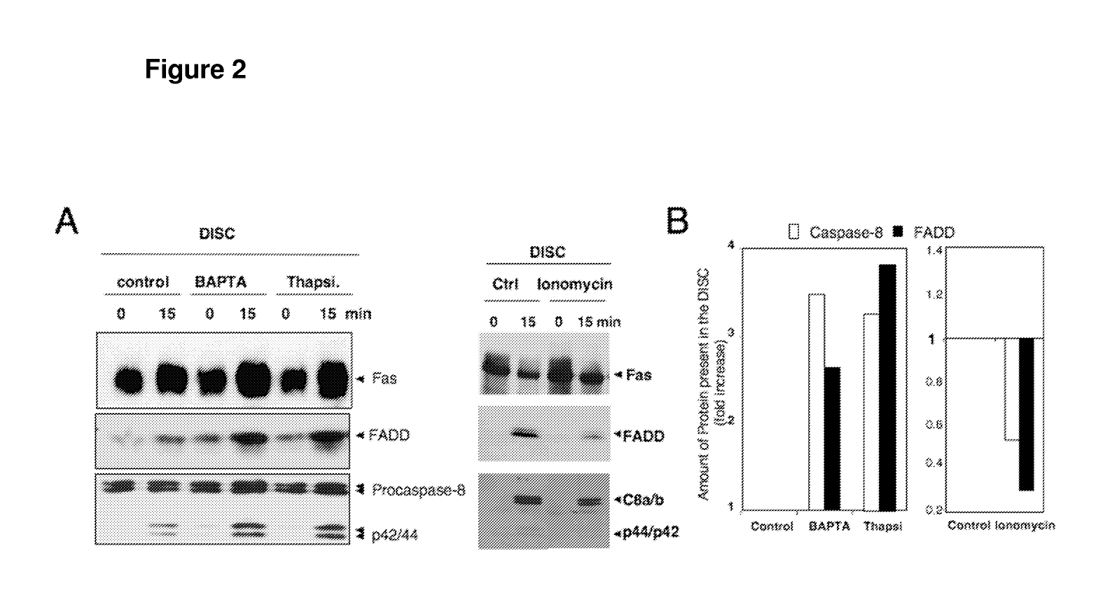

As demonstrated in FIG. 2, the reduction in intracytoplasmic calcium concentration by incubating cells with non-cytotoxic concentrations (1-10 μM) of a calcium chelator such as BAPTA-AM (1,2-bis-(o-Amino phenoxy)ethane-N,N,N′,N′-tetraacetic tetra-(acetoxymethyl) acid) Ester) enhanced formation of the DISC macro-complex as compared to untreated cells. On the contrary, it was demonstrated that the increase in intracellular calcium [Ca²⁺]ᵢ induced by adding ionomycine, a calcium ionophore, interfered with the formation of the DISC complex and as a consequence decreased the FADD binding and the activation of caspase-8. These results evidenced that calcium played a part in the anti-apoptotic function during the early steps of Fas signalling, before the FADD recruitment and formation of DISC.

### Example 9

### Evidence of a Cooperation Between Intracellular Calcium Depletion and Fas Signaling

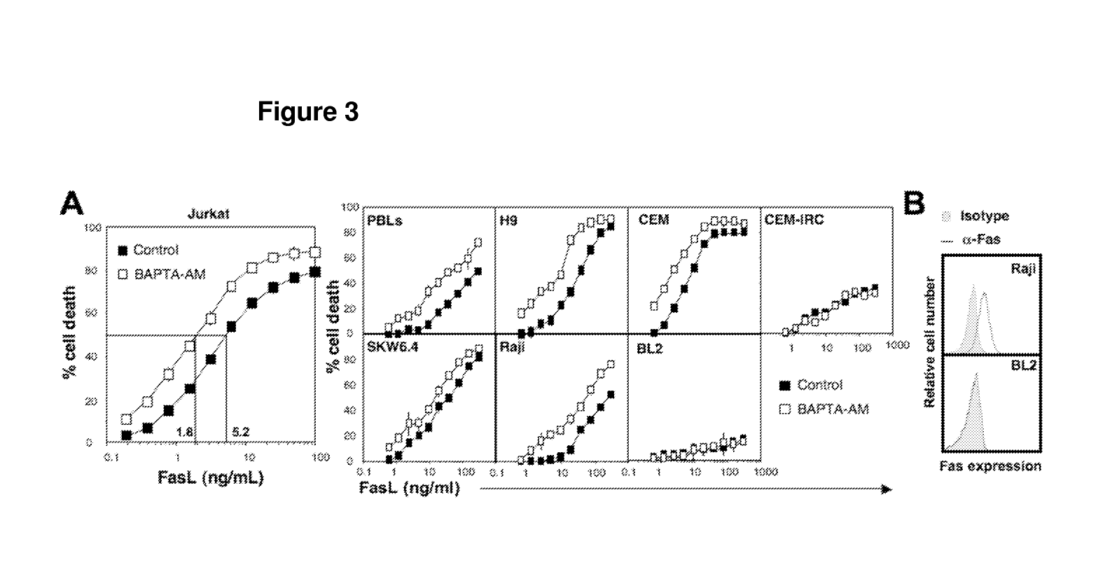

The results presented in FIG. 3A showed that numerous cell lines derived from leukemiae, lymphomae, or activated peripheral blood lymphocytes (activated PBLs, peripheral blood lymphocytes) were sensitised to the Fas-mediated apoptotic signal following treatment using the calcium chelator BAPTA-AM. Conversely, the Burkitt lymphoma BL2 cell line, devoid of endogenous Fas have not been sensitised by this treatment. These results confirm that the combination of an apoptotic inducer triggering the Fas death receptor with an agent capable of reducing the quantity of intracellular calcium enabled significant potentiation of the induction of the apoptosis signal and the elimination of tumour cells.

### Example 10

### Evidence of Potentiating Effect of Store-Operated Channel Blockers on the Fas-Mediated Apoptotic Signal

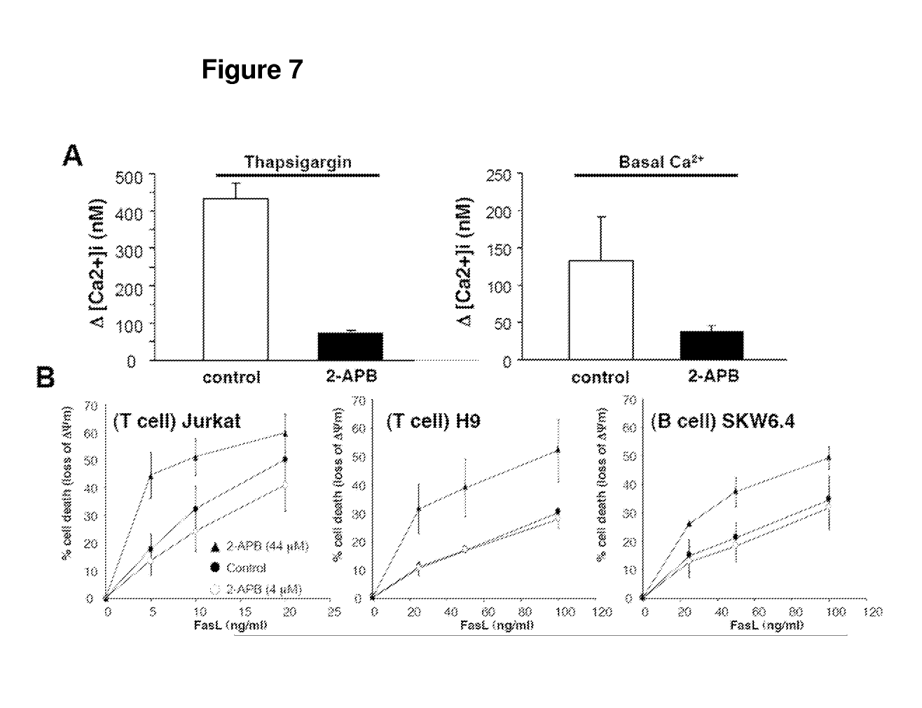

The results showed that the use of high doses of 2-APB inhibiting calcium channels (SOCs), induced decreasing concentration of intracellular calcium and potentiated FasL response in T or B lymphoma cell lines (FIGS. 7A-B).

### Example 11

### Association of Agents Capable of Decreasing Extracellular Calcium and of at Least One Anticancer Agent

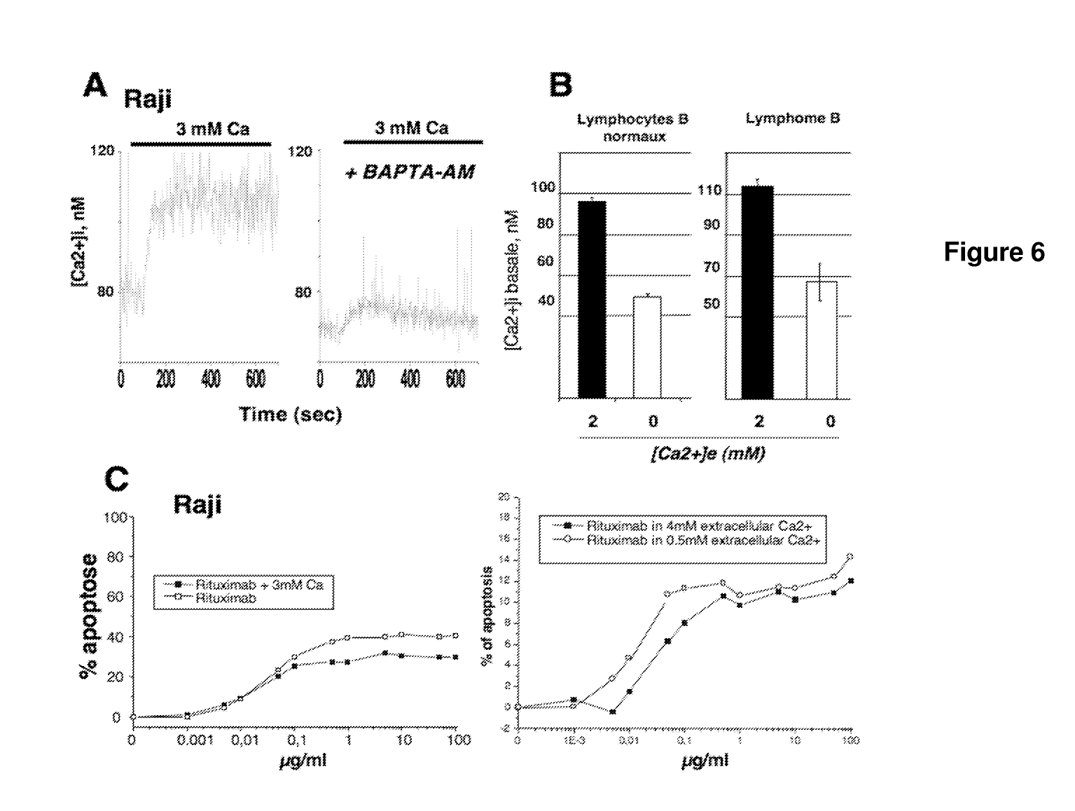

This example aimed to show the effect of extracellular calcium on intracellular calcium concentration was studied by dual wavelength microspectrofluorometry in lymphoma human cells. The results presented on FIGS. 6A and B showed that the changes in extracellular calcium concentrations ([Ca²⁺]ₑ), +/−3 mM had a substantial impact on intracellular calcium concentrations [Ca²⁺]ᵢ. Also, the addition of extracellular calcium inhibited the cell death signal induced by the delivery of Rituximab (FIG. 6C). On the contrary, a medium containing low concentrations of calcium (0.5 mM) allowed potentiating the death signalling and apoptosis induced by Rituximab (FIG. 6D).

These results clearly showed that modulation of [Ca²⁺]ₑ potentiated the mediation of the apoptotic signal mediated by anti-tumoral agents via death receptors.

### Example 12

### Synergistic Association of a Calcium Chelator Agent and of at Least One Anticancer Agent

The results presented in this example show that pre-incubation of B lymphoma cell lines with a calcium chelator such as BAPTA-AM allowed potentiating apoptotic signals mediated by chemotherapeutic agents such as edelfosine or Rituximab (FIG. 4). On the contrary, no increase in the apoptotic signal has been observed when the BL2 cell line, deprived of the Fas receptor and Rituximab resistant, was treated with Rituximab associated with BAPTA-AM. These results confirmed the involvement of the Fas receptor in this apoptotic signalling pathway according to the invention (FIG. 4).

### Example 13

### Potentiation of the Apoptotic Signal Induced by DR4 or DR5 Death Receptor

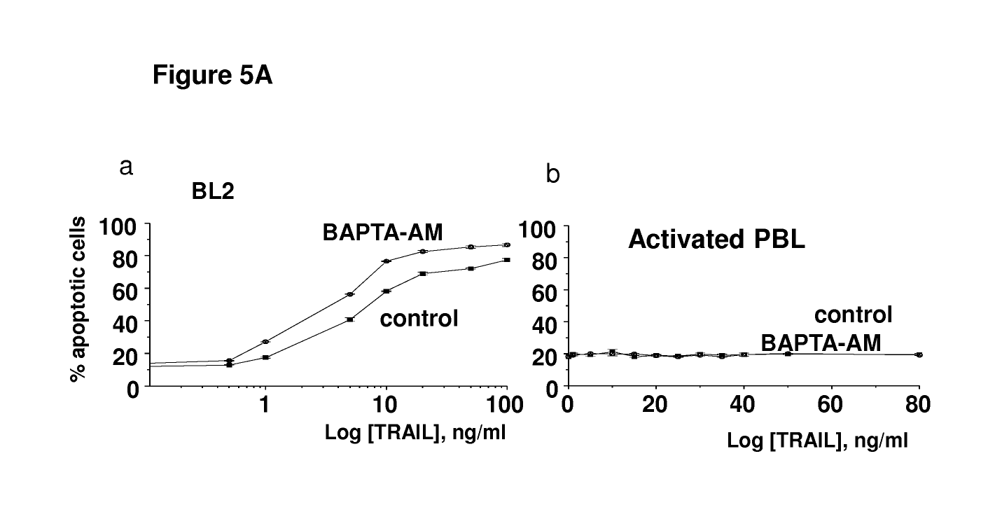

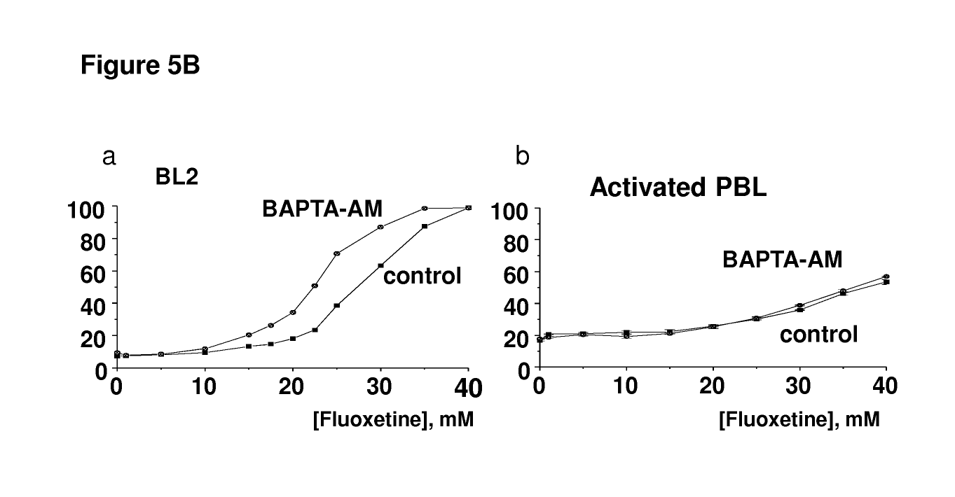

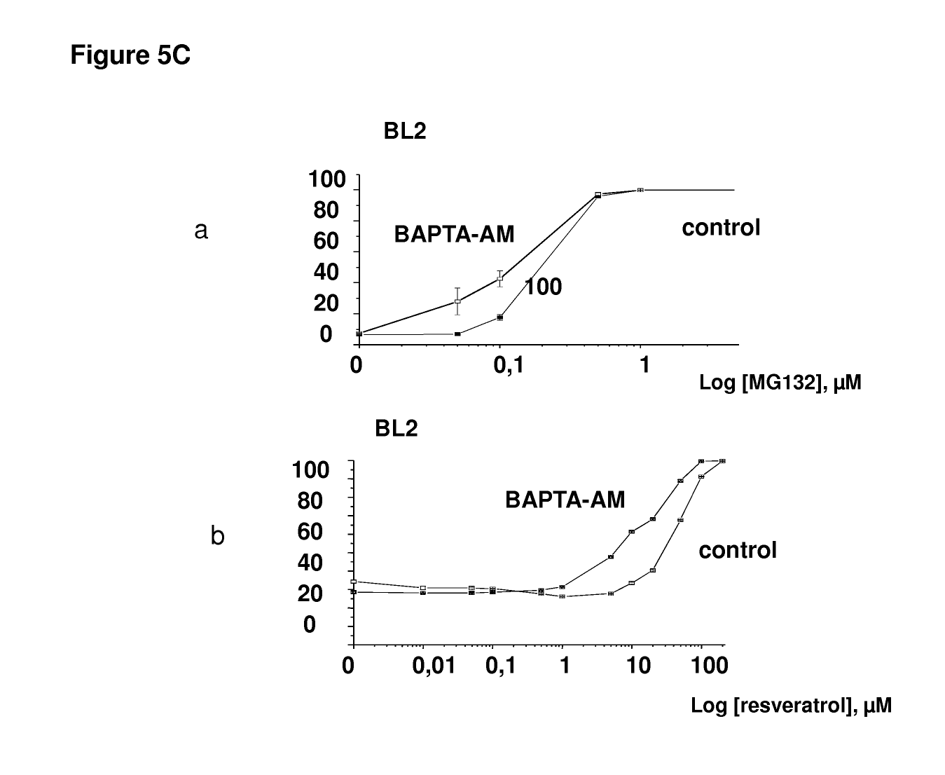

Results as presented in FIGS. 5A-C showed that the induction of cell death by adding the TRAIL ligand (FIG. 5A) or drugs acting via DR4 or DR5 death receptor, such as fluoxetine, MG132 and resveratrol (FIGS. 5B-C) was also potentiated by calcium chelation. Results showed that this effect was specific for tumoral cells since no effect was observed in activated PBL (non tumoral lymphocytes).

### Example 14

### Synergistic Association of a Modulator Agent Capable of Reducing Intracellular Calcium and of a PI3K Inhibitor

The results presented in FIGS. 4C and 4D show that the association of a PI3K signalling pathway inhibitor, edelfosine, with an agent capable of reducing intracellular calcium allowed potentiating the apoptotic signal mediated by edelfosine in tumour cell lines.

### Example 15

### CD95 Stimulation Drives a Rapid Increase in Intracellular Calcium Independently of the DISC Formation

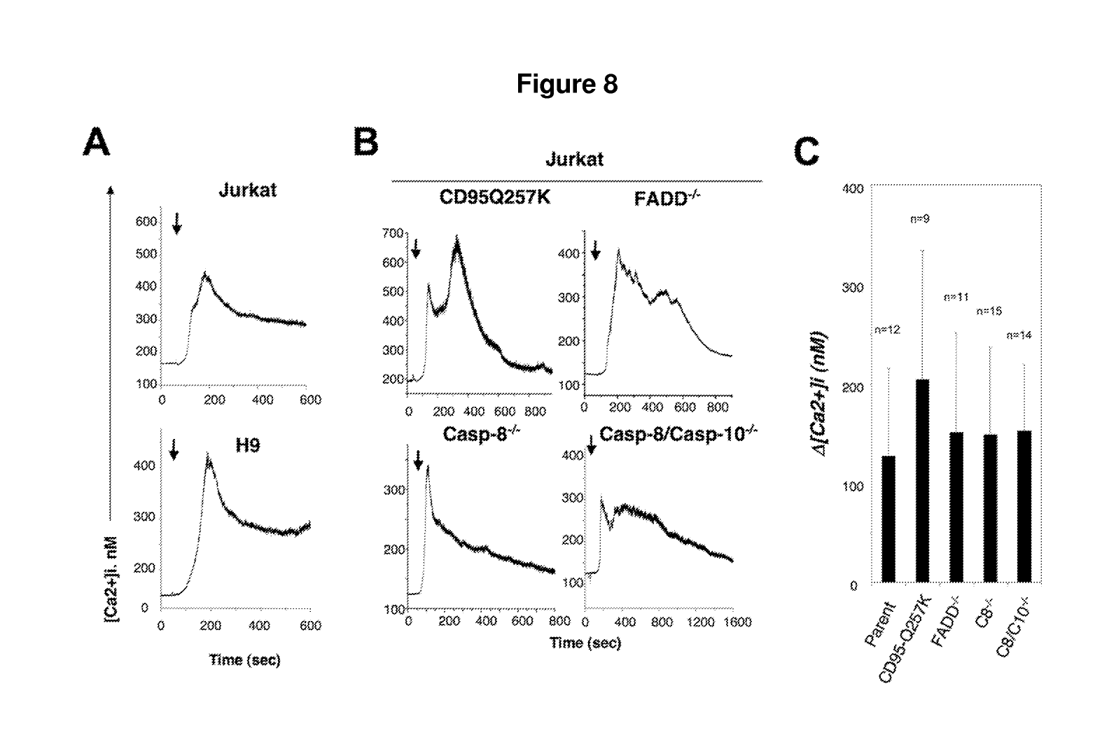

The variation of intracellular calcium upon addition of soluble CD95L was measured in two different leukemic T-cells, the type I cell H9 and the type II cell Jurkat (FIG. 8A) in order to assess whether activation of CD95 alters the intracellular concentration of calcium ([Ca²⁺]ᵢ). Administration of CD95L resulted in a rapid and transient increase in calcium from the basal level comprised between 100 and 150 nM up to 700 nM (FIGS. 8A and 8C).

A leukemic T-cell line expressing a hemizygous mutated allele of CD95 (Jurkat-CD95-Q257K), which displayed resistance towards the CD95 signal was then used to determine whether the peak of calcium relied on the death domain of CD95 and the major components of the DISC (i.e., FADD, caspase-8 and caspase-10). Results showed that although the hemizygous expression of this death domain-mutated allele of CD95 (Q257K) altered both formation of the DISC and transmission of the apoptotic signal, the calcium response remained unaltered in the Jurkat CD95-Q257K as compared to the parental cell line (FIGS. 8A, B and C). Calcium increase was thus originated from a death domain-independent mechanism. To further validate his observation, the role of the major DISC components in the intracellular calcium increase was investigated. Strikingly, while cells devoid of FADD, caspase-8 or caspase-8 and -10 remained insensitive to the CD95-mediated apoptotic signal, they did not display any modification of the increase in [Ca²⁺]ᵢ observed upon addition of CD95L (FIGS. 8A and 8B). These results thus showed that the CD95 death domain and the DISC components did not participate in mounting the calcium response.

### Example 16

### Decrease in Intracellular Calcium Concentration Promoted a Cd95L-Independent Clustering of CD95

Since the peak of calcium occurred independently of the DISC formation and ionic signal occurred rapidly in the cells, it was assessed whether calcium ions may participate in the initial steps of the CD95-mediated apoptotic signal.

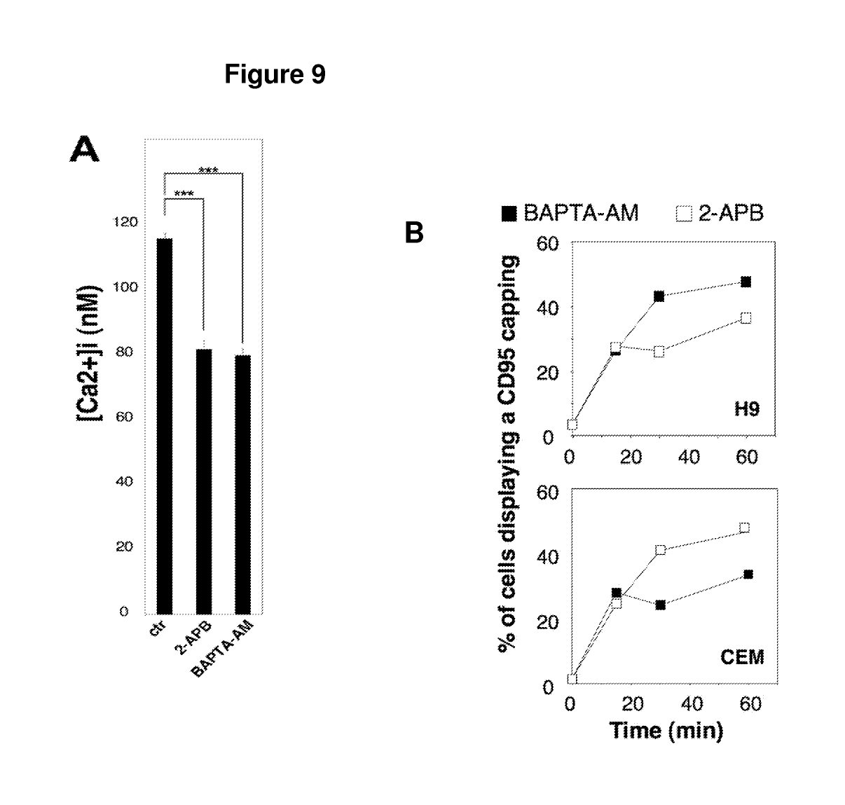

The pre-incubation of cells with either the calcium chelator BAPTA-AM or the blocker of IP3-Receptors and CRAC channels termed 2-Aminoethoxydiphenyl borate (2-APB) induced a faint decrease in the basal [Ca²⁺]ᵢ(FIG. 9A). Strikingly, this decrease in intracellular calcium drove the formation of CD95 capping (CD95-CAP), a pivotal and early step in the induction of CD95 signal. The effect of calcium down-modulation was significant since decrease in [Ca²⁺]ᵢ for 60 minutes promoted the formation of CD95-CAP in up to 50% of the treated cells (FIG. 9B). Although 50% of cells exhibited a CD95-CAP, no trace of caspase-8 activation or mitochondrial depolarization was detected during this treatment, which pointed out that additional apoptosis check-points existed downstream the CD95-CAP formation.

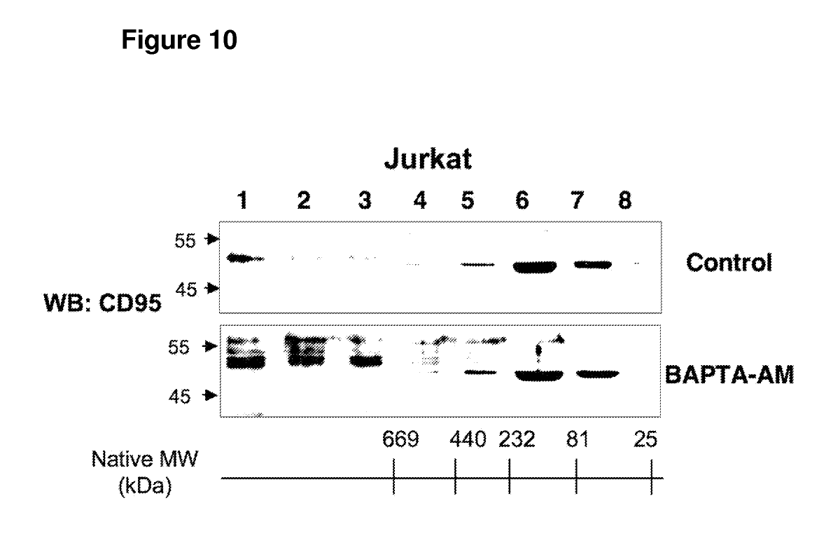

In order to validate that CD95 underwent aggregation upon down-modulation of intracellular calcium, the formation the micrometer-sized CD95-containing domain was investigated by using size exclusion chromatography (FIG. 10). Decrease in intracellular calcium allowed the redistribution of a significant proportion of CD95 into heavy weighted fractions (FIG. 10), which confirmed that the slight down-modulation of [Ca²⁺]ᵢ led to the formation of cluster of CD95. It was also observed that calcium-driven CD95-CAP occurred independently of de novo expression of CD95L since no trace of the membrane-bound ligand was detected. In addition, 2-APB or BAPTA-AM regimens did not modify the expression of CD95. In conclusion, these findings suggested that down-modulation of intracellular calcium facilitated the initial steps of the CD95 signal by promoting the formation of CD95 aggregates. Since CD95 clustering is an early step of the CD95 molecular ordering, the impact of intracellular calcium on the initial events of CD95 pathway and on cell death was further assessed.

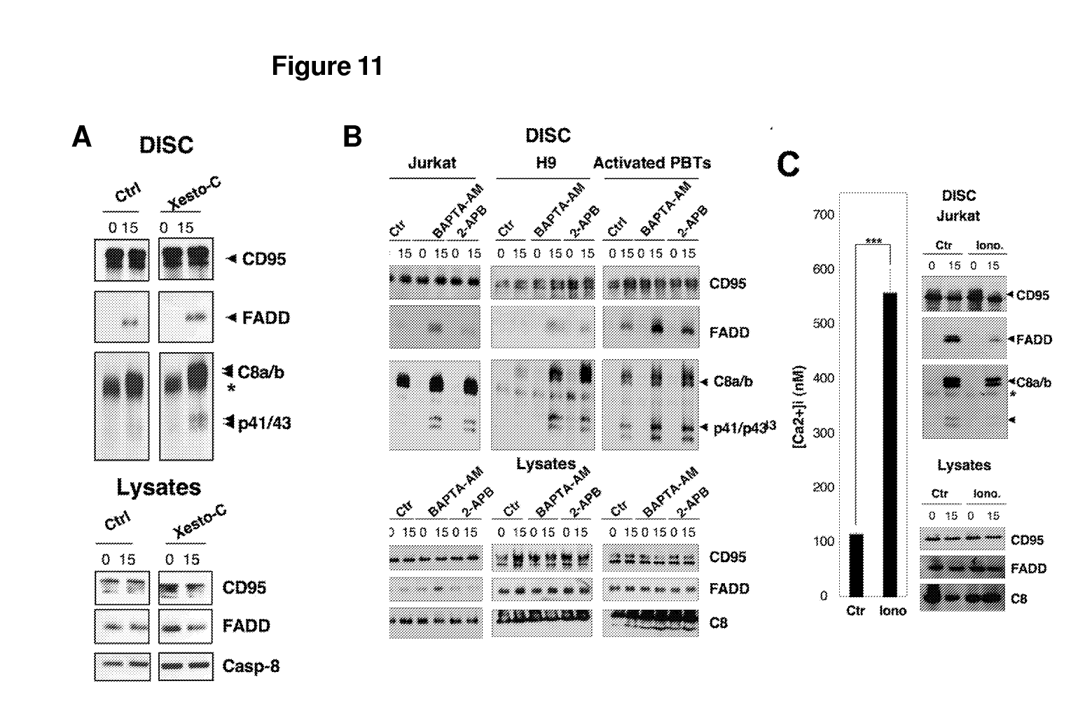

The effect of non-cytotoxic concentrations of BAPTA-AM and 2-APB was determined on the peak of intracellular calcium observed upon CD95 stimulation. Xestospongin C, a marine alkaloid isolated from the sponge Xestospongia species, was used as a potent and selective cell-permeable inhibitor of IP₃ receptor-mediated Ca²⁺ release. Previous studies revealed the essential function of IP3-R in the calcium increased upon CD95 engagement. The role of IP3-R induction and the subsequent release of the intracellular ER-store of calcium was confirmed using xestospongin C, which significantly promoted FADD binding to CD95 (FIG. 11A). Our findings demonstrated that the entry of extracellular calcium was crucial for the calcium peak upon CD95 engagement.

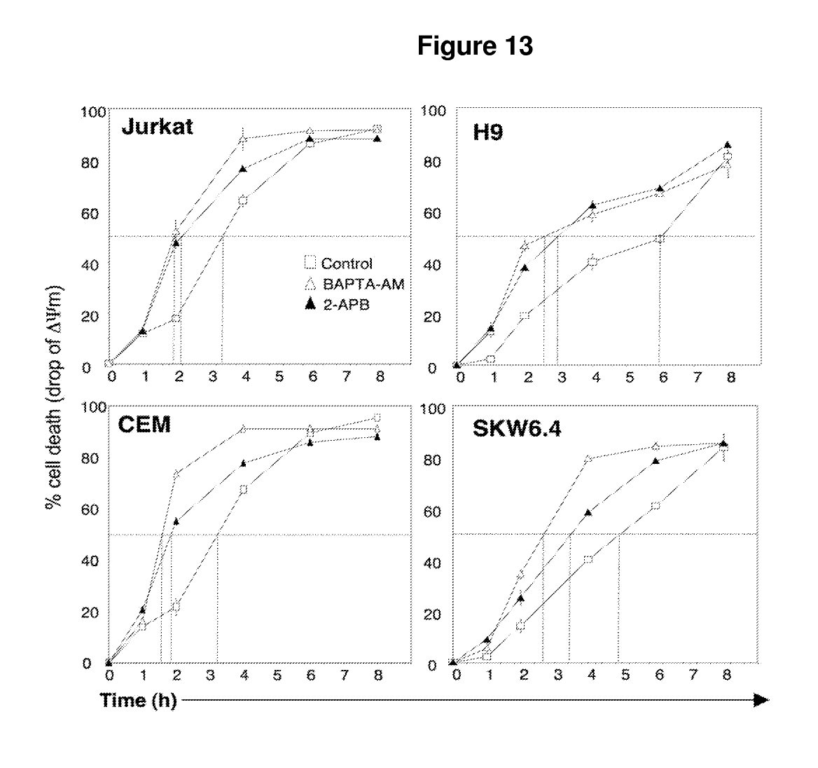

While both BAPTA-AM and 2-APB totally abrogated the CD95-mediated peak of calcium, these chemicals dramatically improved the DISC formation in type I (H9), type II (Jurkat) leukemic T-cells and in activated peripheral blood T-lymphocytes (PBTs) isolated from healthy donors (FIG. 11B). Inhibition of the calcium peak promoted the recruitment of the adaptor protein FADD, which in turn aggregated larger amount of caspase-8 as compared to untreated cells (FIG. 11B). On the other hand, increase in [Ca²⁺]ᵢ upon addition of the calcium ionophore, ionomycin prevented the recruitment of FADD to CD95 and the DISC formation (FIG. 13C).

Altogether these findings strongly supported that intracellular calcium behaves as a potent modulator of the initial steps of the CD95 signal, interfering with the recruitment of the adaptor protein FADD to the CD95 death domain.

### Example 17

### Down-Modulation of Intracellular Calcium Concentration Accelerated the CD95-Mediated Apoptotic Signal

Since down-modulation of the intracellular calcium concentration enhanced the initial events of the CD95 signal, it was next explored whether the processing of initiator caspase-8 and the sensitization of the cells to the CD95-mediated apoptotic signal was enhanced.

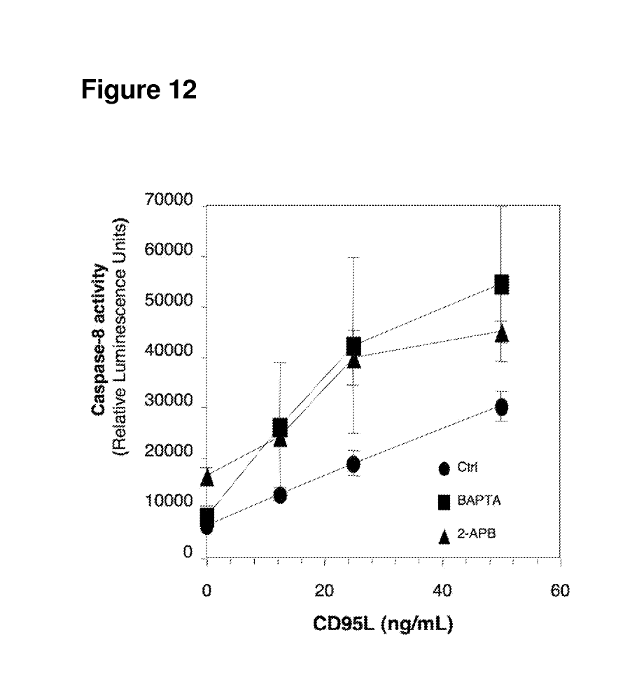

A kinetic of the caspase-8 activation revealed that alteration of the calcium response using either BAPTA-AM or 2-APB accelerated the cleavage of the initiator caspase-8 in T and B-cell lines upon addition of CD95L. The increase in caspase activity upon down-modulation of [Ca²⁺]ᵢ was confirmed by a caspase assay using a proluminogenic substrate of caspase-8 (FIG. 12).

To further ascertain the impact of calcium modulation upon the cell death kinetic, loss mitochondrial potential, an irreversible marker of cell death was assessed. Blockade of the intracellular increase in the calcium concentration significantly accelerated the cell death process in leukemic cells (FIG. 13).

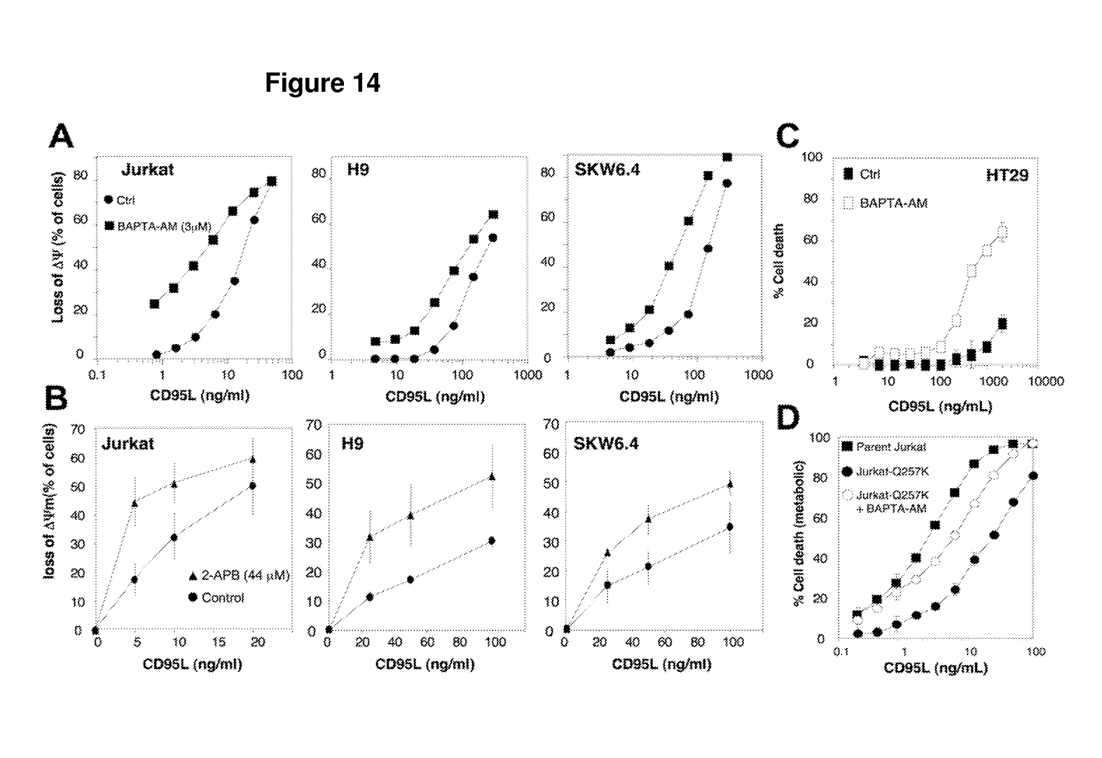

Finally, down-modulation of the intracellular amount of calcium enhanced the CD95-mediated apoptotic signal both in hematologic (FIGS. 14A and 14B) and non-hematologic cell lines (FIG. 14C). Indeed, the colorectal adenocarcinoma cell line HT29, which displays resistance towards the CD95-mediated apoptotic signal was resensitized to death upon the decrease in [Ca²⁺]ᵢ(FIG. 14C). It is thus concluded that down-modulation of the intracellular amount of calcium was likely to decrease the apoptotic threshold required to induce the CD95-mediated apoptotic signal and thus may resensitize to death CD95-resistant cells.

The leukemic T-cell line Jurkat harboring a hemizygous mutated allele of CD95 (Jurkat-CD95Q257K) exhibited resistance towards the CD95 signal (FIG. 14D). Strikingly pre-incubation of this leukemic T-cell with non-cytotoxic doses of BAPTA-AM (FIG. 14D) and 2-APB restored the CD95-mediated apoptotic signal at a level comparable to the parental cell line.

Altogether these findings emphasized that intracellular calcium played a crucial role in the modulation of the initial steps of CD95 signal and its decrease was likely to resensitize to CD95-resistant tumor cells.

### Example 18

### Involvement of Extracellular and Intracellular Pools of Calcium in the Modulation of the Apoptotic Signal CD95

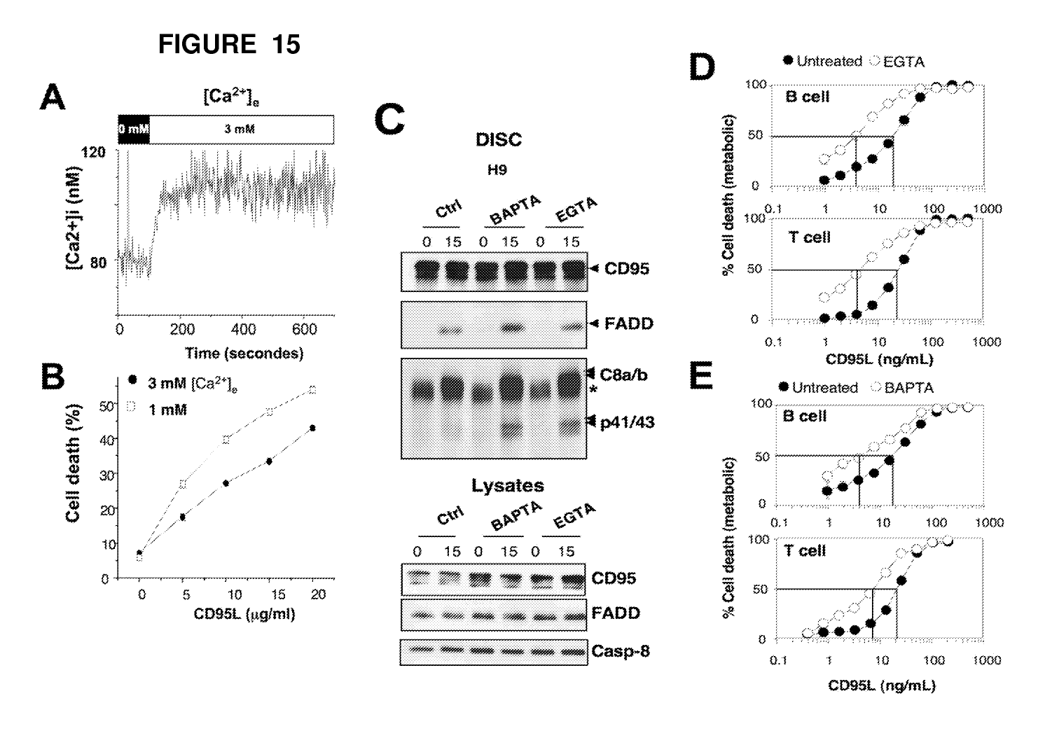

It was next addressed whether both extracellular (medium store) and intracellular (ER store) calcium participate in the modulation of the initial steps of the CD95 signal. Calcium homeostasis was observed to be tightly controlled and an increase in extracellular concentration of calcium ([Ca²⁺]ₑ) resulted in an augmentation of intracellular concentration (FIG. 15A). Furthermore, cells growing in a medium supplemented with calcium (3 mM) resisted to the CD95-mediated apoptotic signal as compared to cells incubated in a medium containing a lower amount of extracellular free calcium (1 mM) (FIG. 15B). Similarly to [Ca²⁺]ᵢ decrease, the down-modulation of the extracellular calcium using both non-permeant BAPTA BAPTA and EGTA enhanced the recruitment of FADD and the DISC formation upon stimulation of CD95 (FIG. 15C).

Finally, down-modulation of [Ca²]ₑ using either EGTA or BAPTA increased the sensitivity of T and B-cell lines to the CD95-mediated apoptotic signal (FIGS. 15D and 15E).

Overall, these findings highlighted the pivotal role of [Ca²]ₑ and [Ca²⁺]ᵢ as potent modulators of the initial steps of the CD95 signal acting prior to the FADD recruitment.

### Example 19

### Involvement of the CRAC channel STIM/ORAI in the Calcium Entry Upon CD95 Stimulation

Store-operated Ca²⁺ entry is mediated by Ca²⁺ release-activated Ca²⁺ (CRAG) channels following Ca²⁺ release from intracellular ER stores. It is known that in activated lymphocytes, CRAG channel corresponded to the ER-calcium sensor STIM-1 and the pore ORAI-1. Since it was showed that both [Ca²⁺]ₑ and [Ca²⁺]ᵢ were involved in the initial steps of the CD95 signaling pathway, it was assumed that entry of calcium may be mediated through the activation of the CRAG channel ORAI-1. When CD95L was added to cell culture, STIM1 moved rapidly from cytosol to a plasma membrane distribution, which is known as a feature of polymerization of ORAI-1 and CRAG activation. Strikingly, the plasma membrane staining of both STIM-1 and ORAI-1 merged with the CD95-CAP, which indicated that the entry of extracellular calcium occurred at the level of the CD95-CAP. Using single-cell imaging of cytosolic Ca²⁺, it was observed that the CD95 stimulation drove a calcium influx displaying a heterogeneous distribution. Indeed, fine analysis of the calcium entry upon CD95 engagement revealed a gradient of [Ca²⁺]ᵢ from the CD95-CAP to the opposite side of the cell.

### Example 20

### Effects of the Hypocalcemia-Inducing Agent Zoledronate In Vitro

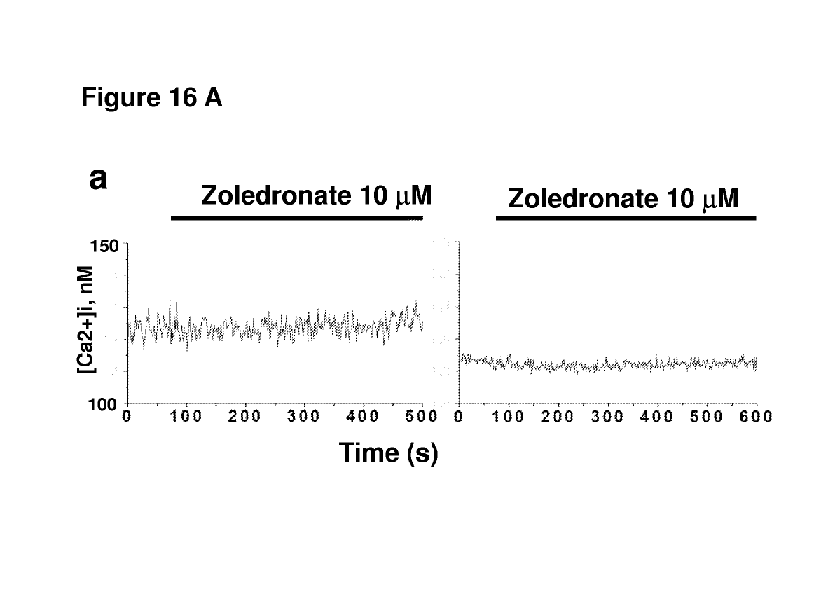

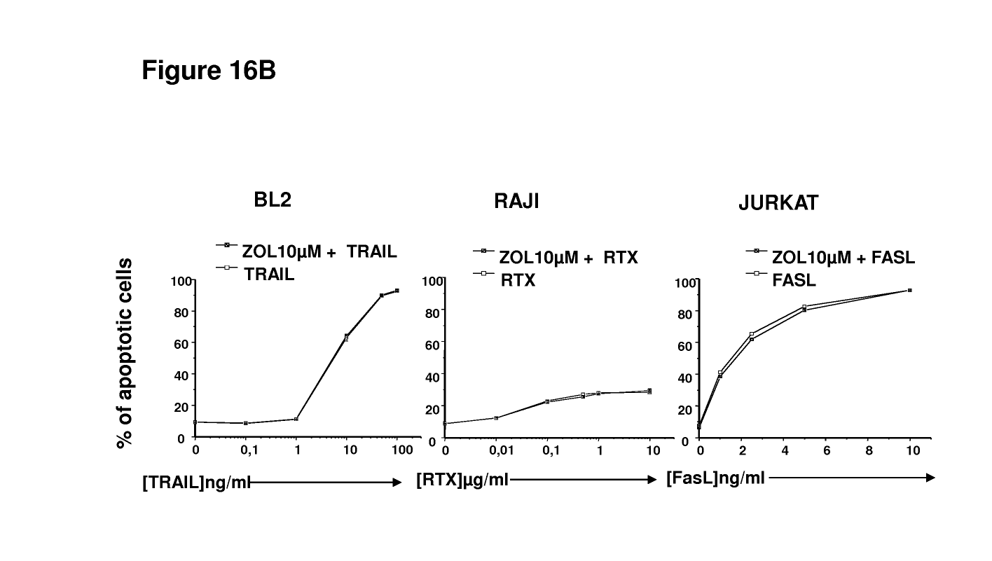

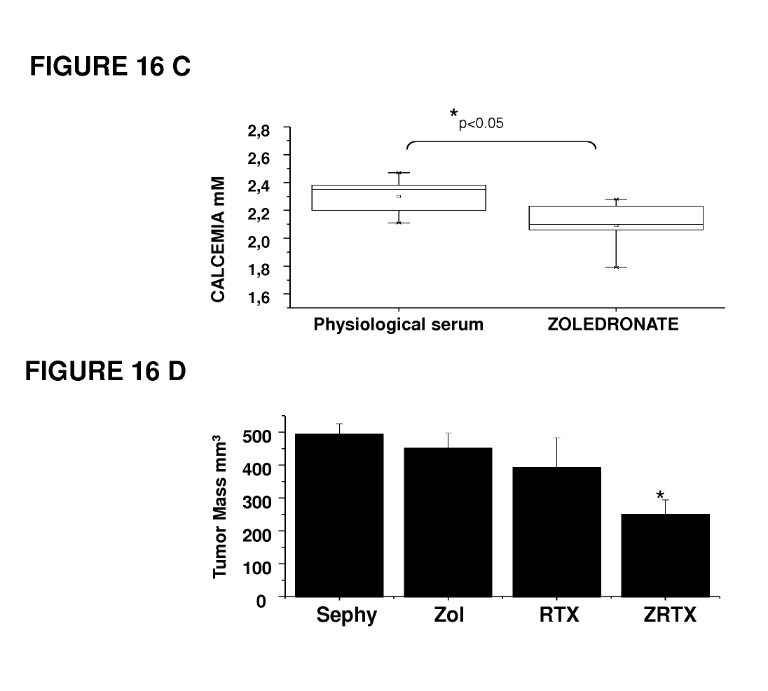

Several studies have shown that zoledronate had direct effects on apoptotic signalling in various cell lines. One of putative mechanism suggested to explain these effects was the calcium chelation by Zoledronate. However, in our experimental conditions, it was showed that Zoledronate did not affect intracellular calcium concentration in all model cell lines tested (FIG. 16 A). In the same way, it was evidenced that in vitro, Zoledronate (10 μM) does not potentiate the apoptotic response to TRAIL, RTX and FasL in the hematopoietic cell lines BL2, Raji and Jurkat, respectively (FIG. 16 B).

### Example 21

### Effect of Zoledronate on Mice Calcemia

Mice treated with Zoledronate injection (one injection per week, for 4 weeks, 4 mg/kg) exhibit a significant (p<0.05) decrease in their calcemia compared to mice treated with vehicle only (physiological serum) (FIG. 16 C). Blood samples were taken from mice 3 days after the last Zoledronate or vehicle injection and calcemia was assessed by COBAS INTEGRA Calcium® (Roche) micro-method.

### Example 22

### Effects of a Rituximab Association with Zoledronate on the Growth of a B Lymphoma Tumour

The implantation of the tumours was done subcutaneously in Rag2−/−γ−/− mice flank aged from 5 to 7 weeks. Following implantation of the tumours, the mice were divided in groups of 8 to 10 animals for each treatment. The size of the tumour was assessed every day by measuring its width (w) and its length (I). Its volume was calculated according to the formula: V=lw²/2. At the end of the experiment, the animals were sacrified, the tumour weight was measured.

From 10⁶ Raji cells were implanted subcutaneously in mice flank. The effects of Rituximab alone, of Zoledronate alone, are assessed on the growth of tumours. Rituximab (2 mg/kg) was delivered 3 times a week, Zoledronate was delivered 1 time a week. Under these conditions, we show that the tumour mass was not significantly affected by treatment of mice with Rituximab or Zoledronate alone compared to treatment with vehicle (physiological serum/Sephy) while the tumour mass of mice treated with Rituximab in association with Zoledronate was significantly (p<0.05) reduced by about 50% (FIG. 16D).

### Example 23

### Effects of Hypocalcemia-Inducing Agents in Association with Anticancer Agents on Tumour Growth: Study in Small Animals

The implantation of the tumours is done subcutaneously in Rag2−/−γ−/− mice flank aged from 5 to 7 weeks. Following implantation of the tumours, the mice are divided in groups of 8 to 10 animals for each treatment. The size of the tumour is assessed every day by measuring its width (w) and its length (I). Its volume is calculated according to the formula: V=lw²/2. At the end of the experiment, the animals are sacrified, the tumour weight is measured.

Zoledronate and BAPTA-AM are used as hypocalcemia-inducing agents. Zoledronate and BAPTA-AM are delivered following the experimental paradigm and in the efficient concentration to decrease significantly calcemia before implantation of the tumour and for the whole duration of the treatment with the anticancer agent of interest.

For each substance tested, the reactants corresponding to the vehicle(s) of the active substance(s) are tested on the tumour growth of a group of animals designated as “control group”.

Model 1: Effects of a Soluble TRAIL (Killer TRAIL, Alexis) Association with Zoledronate or BAPTA-AM on the Growth of a B Lymphoma Tumour.

From 10⁶ to 10⁷ BL2 cells are implanted subcutaneously in mice flank. The effects of soluble TRAIL alone, of Zoledronate alone, of BAPTA-AM alone are assessed on the growth of tumours. Soluble TRAIL is delivered according to the protocols known by the man of the art, Zoledronate, BAPTA-AM are delivered according to the protocol described above. Under these conditions, tumour growth is likely to decrease from: 0 to 50%; 5 to 50%; 10 to 50%; 15 to 50%; 20 to 50%; 25 to 50%; 30 to 50%; 35 to 50%; 35 to 50%; 40 to 50%; 45 to 50%. When soluble TRAIL is associated with Zoledronate or BAPTA-AM, tumour growth is likely to decrease from: 10 to 90%; 15 to 90%, 20 to 90%; 25 to 90%; 30 to 90%; 35 to 90%; 40 to 90%; 45 to 90%; 50 to 90%; 55 to 90%; 60 to 90%; 65 to 90%; 70 to 90%; 75 to 90%; 80 to 90%; 85 to 90%.

Model 2: Effects of Fluoxetine Association with Zoledronate or BAPTA-AM on the Growth of a B Lymphoma Tumour and Colorectal Tumour Cell Line HCT116

From 10⁶ to 10⁷ BL2 cells or HCT116 ells are implanted subcutaneously in mice flank. The effects of fluoxetine alone, of Zoledronate alone, of BAPTA-AM alone are assessed on the growth of tumours. Fluoxetine is delivered according to the protocols known by the man of the art, Zoledronate, BAPTA-AM are delivered according to the protocol described above. Under these conditions, tumour growth is likely to decrease from: 0 to 50%; 5 to 50%; 10 to 50%; 15 to 50%; 20 to 50%; 25 to 50%; 30 to 50%; 35 to 50%; 35 to 50%; 40 to 50%; 45 to 50%. When fluoxetine is associated with Zoledronate or BAPTA-AM, tumour growth is likely to decrease from: 10 to 90%; 15 to 90%, 20 to 90%; 25 to 90%; 30 to 90%; 35 to 90%; 40 to 90%; 45 to 90%; 50 to 90%; 55 to 90%; 60 to 90%; 65 to 90%; 70 to 90%; 75 to 90%; 80 to 90%; 85 to 90%.

1-15. (canceled)

16. A composition for treating cancer, potentiating formation of DISC (Death Inducing Signalling Complex) macro-complex, or inducing apoptotic signal mediated by death receptors in tumor cells, the composition comprises (i) a therapeutically effective amount of an active agent selected from the group consisting of a hypocalcemia-inducing agent, a calcium channel inhibitor, and a calcium chelator, and (ii) a therapeutically effective amount of an anticancer agent capable of inducing an apoptotic signal via death receptors Fas, TNF-R1, DR4 or DR5.

17. The composition of claim 16, wherein the hypocalcemia-inducing agent is a biphosphonate, a calcimimetic agent or calcitonine.

18. The composition of claim 16, wherein said calcium chelator is BAPTA, EGTA, EDTA, CDTA, a permeant form thereof, BAPTA-AM, EGTA-AM, MAPTA-AM, 5,5′ F2 BAPTA, or cardioxan.

19. The composition of claim 17, wherein said calcimimetic agent is cinacalcet.

20. The composition of claim 16, wherein said calcium channel inhibitor is 2-Aminoethoxydiphenyl borate (2-APB), ML-9 or BTP2.

21. The composition of claim 17, wherein said biphosphonate is palmidronate, zoledronate, etidronate, ibandronate or clodronate.

22. The composition of claim 16, wherein said anticancer agent is present in a sufficient amount to induce an apoptotic signal mediated by the death receptors, and is selected from the group consisting of an anti-CD20 antibody, an anti-Fas antibody, an anti-TNF-R1 antibody, an anti-DR4 antibody, an anti-DR5 antibody, FasL, TRAIL, and soluble or multimeric soluble forms thereof.

23. The composition of claim 22, wherein said anti-CD20 antibody is Rituximab, Rituxan®, GA-101, ofatumab, LFB-R603, or Veltuzumab.

24. The composition of claim 16, wherein the tumor cells are selected from the group consisting of colon cancer cells, breast cancer cells, prostate cancer cells, lung cancer cells, ovary cancer cells, pancreas cancer cells, kidney cancer cells, brain cancer cells, sarcomas, testis cancer cells, lymphoma, liver cancer cells, and B lymphoma cells.

25. A method of treating cancer or preventing cancer relapses in a patient, comprising the step of administering a therapeutically effective amount of the composition of claim 16 to said patient.

26. The method of claim 25, wherein said treated patients are affected by primary tumour, hematopoietic cancer or solid tumor, without the occurrences of bone metastasis.

27. The method of claim 25, wherein the active agent is administered in a therapeutically effective amount which is lower than the amount currently administered for treating metabolic diseases associated with neoplasic diseases.

28. A method of screening for compounds capable of potentiating the pro-apoptotic effect of an anticancer agent comprising the step of contacting said compounds to tumor cells and assessing the formation of DISC macro-complex by selective potentiation.

29. The method of screening according to claim 28, wherein pro-apoptotic potentiating effect is assessed by measuring mitochondrial transmembrane electrical potential, membrane permeability, DNA fragmentation, cell morphology, Western blot, or measuring caspase activity.
